(19) United States
(12) Patent Application Publication
(10) Pub. No.: US 2018/0064765 A1
(43) Pub. Date: Mar. 8, 2018

(54) LISTERIA-BASED IMMUNOGENIC COMPOSITIONS FOR ELICITING ANTI-TUMOR RESPONSES

(71) Applicants: Advaxis, Inc., Princeton, NJ (US); The United States of America, as represented by The Secretary, Department of Health and Human Serv, Bethesda, MD (US)

(72) Inventors: Robert PETIT, Newtown (Wrightstown), PA (US); Anu Wallecha, Yardley, PA (US); Samir Khleif, Silver Springs, MD (US); Zhisong Chen, Potomac, MD (US); Jay A. Berzofsky, Bethesda, MD (US)

(21) Appl. No.: 15/326,011

(22) PCT Filed: Jul. 17, 2015
(86) PCT No.: PCT/US15/40922

§ 371 Date: Jan. 12, 2017

Related U.S. Application Data
(60) Provisional application No. 62/026,221, filed on Jul. 18, 2014.

(51) Int. Cl. A61K 35/74; A61K 39/00; A61K 39/12; A61K 39/395; C07K 16/28

(52) U.S. Cl. CPC A61K 35/74; A61K 39/0011; A61K 39/12; A61K 39/3955; C12N 2710/20034; A61K 2039/505; A61K 2039/585; C07K 2317/76; C07K 16/2818

(57) ABSTRACT

The present invention is directed to compositions comprising an immune checkpoint inhibitor or a T cell stimulator or a combination thereof, and a live attenuated recombinant Listeria strain comprising a fusion polypeptide comprising a truncated Listeriolysin O protein, a truncated ActA protein, or a PEST amino acid sequence fused to a tumor-associated antigen. The invention is further directed to methods of treating, protecting against, and inducing an immune response against a tumor or a cancer, comprising the step of administering the same.

### GOVERNMENT INTEREST

This invention was supported, in part, by a Cooperative Research and Development Agreement (CRADA) #02648. The U.S. government may have certain rights in the invention.

### FIELD OF INVENTION

The present invention is directed to compositions comprising an immune checkpoint inhibitor or a T cell stimulator, and a live attenuated recombinant Listeria strain comprising a fusion protein of a truncated Listeriolysin O protein, a truncated ActA protein, or a PEST amino acid sequence fused to a tumor-associated antigen. The invention is further directed to methods of treating, protecting against, and inducing an immune response against a tumor, comprising the step of administering the same.

### BACKGROUND OF THE INVENTION

Listeria monocytogenes (Lm) is a Gram-positive facultative intracellular pathogen that causes listeriolysis. Once invading a host cell, Lm can escape from the phagolysosome through production of a pore-forming protein listeriolysin O (LLO) to lyse the vascular membrane, allowing it to enter the cytoplasm, where it replicates and spreads to adjacent cells based on the mobility of actin-polymerizing protein (ActA). In the cytoplasm, Lm-secretedproteins are degraded by the proteasome and processed into peptides that associate with MHC class I molecules in the endoplasmic reticulum. This unique characteristic makes it a very attractive cancer vaccine vector in that tumor antigens can be presented with MHC class I molecules to activate tumor-specific cytotoxic T lymphocytes (CTLs).

One of several mechanisms of tumor-mediated immune suppression is the expression of T-cell co-inhibitory molecules by tumor. Upon engagement to their ligands these molecules can suppress effector lymphocytes in the periphery and in the tumor microenvironment.

D-1 is expressed on the surface of activated lymphocytes and myeloid cells. PD-L1 is expressed on activated T cells, B cells, dendritic cells and macrophages, in addition to a wide range of non-hematopoietic cells. PD-L1 is upregulated on numerous human tumors, and its expression has been shown to inversely correlate with survival in different types of cancer. The expression of PD-L2 on various tumor cells has also been demonstrated.

It has been shown that tumor eradication can be enhanced by blockade of PD-L1/PD-1 interaction. Recently it has been demonstrated that the combination of PD-1 targeting with vaccine and low-dose cyclophosphamide significantly enhances antigen-specific immune responses, decreases tumor burden and increases survival of treated mice. Interestingly, infection with Listeria leads to up-regulation of PD-L1 on immune cells.

Presently, there remains a need to provide effective combinatorial tumor targeting methods that can eliminate tumor growth and cancer. The present invention addresses this need by providing a combination of Listeria based vaccine with blockade of PD-1/PD-L interaction. As seen in the Detailed Description below, this combination may improve the overall anti-tumor efficacy of immunotherapy.

### SUMMARY OF THE INVENTION

In one aspect, the invention relates to an immunogenic composition comprising an immune checkpoint inhibitor and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In a related aspect, the invention relates to an immunogenic composition comprising a T-cell stimulator, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof.

In another aspect, the invention relates to an immunogenic composition comprising an immune checkpoint inhibitor, a T-cell stimulator, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof.

In one embodiment, the invention relates to an immunogenic composition comprising a programmed cell death receptor-1 (PD-1) signaling pathway inhibitor, or a CD-80/86CTLA4 signalling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1H. LmddA-LLO-E7 induces regression of established TC-1 tumors accompanying with Treg frequency decrease. C57BL6 mice were inoculated s.c. with 1×10⁵ TC-1 tumor cells each, and were immunized i.p. with 0.1 LD50 LmddA-LLO-E7 (1×10⁸ CFU), Lm-E7 (1×10⁶ CFU), or LmddA-LLO (1×10⁸ CFU) in PBS (100 al) on day 10 and day 17 post tumor challenge. Tumor was measured twice a week using an electronic caliper. Tumor volume was calculated by the formula: length×width×width/2. Mice were sacrificed when tumor diameter reached approximately 2.0 cm or on day 24 for Flow cytometric analysis. (FIG. 1A) Average tumor volume from day 10 to day 24. (FIG. 1B) Tumor volume on day 24. (FIG. 1C) Survival percentage. (FIG. 1D) Flow cytometric profile of CD4+FoxP3+ T cells out of CD4+ T cells. (FIG. 1E) Percentage of CD4+FoxP3+ T cells out of CD4+ T cells in the spleen. (FIG. 1F) Ratio of CD4+FoxP3+ T cells to CD8+ T cells in the spleen. (FIG. 1G) Percentage of CD4+FoxP3+ T cells out of CD4+ T cells in the tumor. (FIG. 1H) Ratio of CD4+FoxP3+ T cells to CD8+ T cells in the tumor. Data are presented as Mean±SEM. *P<0.05, **P<0.01, and ***P<0.001 (Mann-Whitney test). Data are from 3 independent experiments (FIG. 1A and FIG. 1B) and are representative of 3 independent experiments (FIGS. 1C-1H).

FIGS. 2A-2D. LmddA-LLO-E7 induces regression of established TC-1 tumors. C57BL6 mice were inoculated s.c. with 1×10⁵ TC-1 tumor cells each, and were immunized i.p. with 0.1 LD50 LmddA-LLO-E7 (1×10⁸ CFU), Lm-E7 (1×10⁶ CFU), or LmddA-LLO (1×10⁸ CFU) in PBS (100 μl) on day 10 and day 17 post tumor challenge. Tumor was measured twice a week using an electronic caliper. Tumor volume was calculated by the formula: length×width×width/2. (FIG. 2A) PBS. (FIG. 2B) LmddA. (FIG. 2C) Lm-E7. (FIG. 2D) LmddA-LLO-E7. Data are from 3 independent experiments.

FIGS. 3A-3E. LmddA-LLO-E7 and Lm-E7 induce similar E7-specific CD8+ T cell response. C57BL6 mice were inoculated s.c. with 1×10⁵ TC-1 tumor cells each, and were immunized i.p. with 0.1 LD₅₀ LmddA-LLO-E7 (1×10⁸ CFU), LmddA-LLO (1×10⁸ CFU), LmddA (1×10⁸ CFU), Lm-E7 (1×10⁶ CFU), or 0.5 LD₅₀ wild-type Lm 10403S (1×10⁴ CFU) in PBS (100 μl) on day 10 and day 17 post tumor challenge. Mice were sacrificed at day 24 and lymphocytes isolated from the spleen and tumor were analyzed by Flow cytometry. FIG. 3A. Flow cytometric prolife of H-2D^{b} E7 tetramer+CD8+ T cells out of CD8+ T cells in the spleen and tumor. (FIG. 3B and FIG. 3C) Percentage of H-2D^{b} E7 tetramer+CD8+ T cells out of CD8+ T cells in the spleen (FIG. 13B) and tumor (FIG. 3C). (FIG. 3D and FIG. 3E) H-2D^{b} E7 tetramer+CD8+ T cell number per mouse spleen (FIG. 3D) and per million tumor cells (FIG. 3E). n=3-10. Data are representative of 3 independent experiments.

FIGS. 4A-4E. L. monocytogenes is sufficient to induce decrease of Treg frequency. C57BL6 mice were inoculated s.c. with 1×10⁵ TC-1 tumor cells each, and were immunized i.p. with 0.1 LD₅₀ LmddA (1×10⁸ CFU) or 0.5 LD₅₀ wild-type Lm 10403S (1×10⁴ CFU) in PBS (100 μl) on day 10 and day 17 post tumor challenge. Mice were sacrificed at day 24 and lymphocytes isolated from the spleen and tumor were analyzed by Flow cytometry. (FIG. 4A) Flow cytometric profile of CD4+FoxP3+ T cells out of CD4⁺ T cells. (FIG. 4B) Percentage of CD4+FoxP3+ T cells out of CD4+ T cells in the spleen. (FIG. 4C) Ratio of CD4+FoxP3+ T cells to CD8+ T cells in the spleen. (FIG. 4D) Percentage of CD4+FoxP3+ T cells out of CD4+ T cells in the tumor. (FIG. 4E) Ratio of CD4+FoxP3+ T cells to CD8+ T cells in the tumor. Data are presented as Mean±SEM. *P<0.05, **P<0.01, and ***P<0.001 (Mann-Whitney test). Data are representative of 3 independent experiments.

FIG. 5. L. monocytogenes decreases Treg frequency by preferentially inducing CD4+FoxP3− T cell and CD8+ T cell expansion. C57BL6 mice were inoculated s.c. with 1×10⁵ TC-1 tumor cells each, and were immunized i.p. with 0.1 LD₅₀ LmddA-LLO-E7 (1×10⁸ CFU), LmddA-LLO (1×10⁸ CFU), LmddA (1×10⁸ CFU), Lm-E7 (1×10⁶ CFU), or 0.5 LD50 wild-type Lm 10403S (1×10⁴ CFU) in PBS (100 μl) on day 10 and day 17 post tumor challenge. Mice were sacrificed at day 24 and lymphocytes isolated from the tumor were analyzed by Flow cytometry. Data are presented as (Mean±SEM). n=3-10. *P<0.05, **P<0.01 (Mann-Whitney test). Data are representative of 3 independent experiments.

FIGS. 6A-6D. L. monocytogenes-induced expansion of CD4+FoxP3− T cells and CD8+ T cells is dependent on and mediated by LLO. C57BL6 mice were injected i.p. with 1×10⁴ CFU 10403S, Δhly, Δhly::pfo, or hly::Tn917-lac (pAM401-hly) in PBS (100 μl). Mice were sacrificed on day 7 post injection and lymphocytes isolated from the spleen were analyzed by Flow cytometry. (FIG. 6A) T cell number in the spleen. (FIG. 6B) Flow cytometric prolife of CD4+FoxP3+ T cells out of CD4+ T cells. (FIG. 6C) Percentage of CD4+FoxP3+ T cells out of CD4+ T cells. (FIG. 6D) Ratio of CD4+FoxP3+ T cells to CD8+ T cells. *P<0.05 (Mann-Whitney test). Data are representative of 3 independent experiments.

FIGS. 7A-7G. Episomal expression of a truncated LLO in LmddA induces expansion of CD4+FoxP3− T cells and CD8+ T cells to a higher level. C57BL6 mice were injected i.p. with 1×10⁸ CFU LmddA or LmddA-LLO in PBS (100 al). Mice were sacrificed on day 7 post injection and lymphocytes isolated from the spleen were analyzed by Flow cytometry. (FIG. 7A) T cell number in the spleen. (FIG. 7B) Flow cytometric prolife of CD4+FoxP3+ T cells out of CD4+ T cells. (FIG. 7C) Percentage of CD4+FoxP3+ T cells out of CD4+ T cells. (FIG. 7D) Ratio of CD4+FoxP3+ T cells to CD8+ T cells. (FIG. 7E) Flow cytometric prolife of Ki-67+ T cells. (FIG. 7F) Percentage of Ki-67+ T cells. (FIG. 7G) Fluorescent intensity of Ki-67+ T cells. (FIG. 7H) Level of Ki-67 expression in CD4+FoxP3− T cell and CD8+ T cells in the presence of LmddA and LmddA-LLO, and control-no vector (PBS). Data are presented as Mean±SEM. *P<0.05, **P<0.01, and ***P<0.001 (Mann-Whitney test). Data are representative of 3 independent experiments.

FIGS. 8A-8G. Combination of Lm-E7 and LmddA-LLO induces regression of established TC-1 tumors. C57BL/6 mice were inoculated s.c. with 1×10⁵ TC-1 tumor cells each, and were immunized i.p. with 0.05 LD₅₀ Lm-E7 (5×10⁵ CFU), 0.05 LD₅₀ LmddA-LLO (5×10⁷ CFU), 0.05 LD50 Lm-E7 plus 0.05 LD₅₀ LmddA-LLO in PBS (100 al) on day 10 and day 17 post tumor challenge. Tumor was measured twice a week using an electronic caliper and tumor volume was calculated by the formula: length×width×width/2. Mice were observed for survival or sacrificed on day 24 and lymphocytes isolated from the spleen were analyzed by Flow cytometry. (FIG. 8A) Average tumor volume from day 10 to day 24. (FIG. 8B) Tumor volume on day 24. (FIG. 8C) Survival percentage. (FIG. 8D) T cell number in the spleen. (FIG. 8E) Flow cytometric prolife of CD4+FoxP3+ T cells out of CD4+ T cells. (FIG. 8F) Percentage of CD4+FoxP3+ T cells out of CD4+ T cells. (FIG. 8G) Ratio of CD4+FoxP3+ T cells to CD8+ T cells. Data are presented as Mean±SEM. *P<0.05, **P<0.01, and ***P<0.001 (Mann-Whitney test). Data are representative of 2 independent experiments.

FIGS. 9A-9G. Adoptive transfer of Tregs compromises the anti-tumor efficacy of LmddA-LLO-E7 against established TC-1 tumors. C57BL6 mice (11 weeks old) were injected s.c. with 1×10⁵ TC-1 tumor cells each, and i.v. with CD4+CD25+ Tregs (1×10⁶ cells/each) on day 9 post tumor challenge. Mice were immunized i.p. with 0.1 LD₅₀ LmddA-LLO-E7 (1×10⁸ CFU) in PBS (100 μl) on day 10 and day 17 post tumor challenge. Tumor was measured twice a week using an electronic caliper and tumor volume was calculated by the formula: length×width×width/2. Mice were sacrificed on day 24 and lymphocytes isolated from the spleen were analyzed by Flow cytometry. (FIG. 9A) Average tumor volume from day 10 to day 24. (FIG. 9B) Tumor volume on day 24. (FIG. 9C) Flow cytometric prolife of CD4+FoxP3+ T cells out of CD4+ T cells. (FIG. 9D) Percentage of CD4+FoxP3+ T cells out of CD4+ T cells in the spleen. (FIG. 9E) Percentage of CD4+FoxP3+ T cells out of CD4+ T cells in the tumor. (FIG. 9F) T cell number in the spleen. (FIG. 9G) T cell number per million tumor cells. Data are presented as Mean±SEM. *P<0.05, **P<0.01, and ***P<0.001 (Mann-Whitney test). Data are representative of 2 independent experiments.

FIG. 10. LmddA does not augment Lm-E7 anti-tumor activity. C57BL/6 mice were inoculated s.c. with 1×10⁵ TC-1 tumor cells each, and were immunized i.p. with 0.05 LD₅₀ Lm-E7 (5×10⁵ CFU), 0.05 LD₅₀ LmddA (5×10⁷ CFU), or 0.05 LD₅₀ Lm-E7 plus 0.05 LD₅₀ LmddA in PBS (100 μl) on day 10 and day 17 post tumor challenge. Tumor was measured using an electronic caliper and tumor volume was calculated by the formula: length×width×width/2. Shown are tumor volumes on day 24. Data are presented as Mean±SEM.

FIGS. 11A-11B. Lm-LLO and Lm-LLO-E7 infection upregulates PD-L1 expression on mouse DC surface. (FIG. 11A) Fold increase of PD-L1 expression on bone marrow derived mouse DC after treatment with different concentrations of Lm-LLO or Lm-LLO-E7 over non-treated control. (FIG. 11B) Representative histogram from one out of three independent experiments.

FIG. 12. Addition of anti-PD-1 Ab to Lm-LLO-E7 enhances therapeutic potency of treatment. A. Treatment schedule. B. Tumor volumes of individual mice for each treatment measured every 3-4 days. C. The Kaplan-Meier plot depicts overall survival. Similar results were obtained from three independent experiments.

FIG. 13. Addition of anti-PD-1 Ab to Lm-LLO-E7 enhances antigen-specific immune responses and increases the level of tumor-infiltrated CD8 T cell. C57BL/6 mice (n=5 per group) were treated as on FIG. 2A, except on day 21 after tumor implantation mice were sacrificed. A. IFN-γ production in the presence or absence of E7 peptide was analyzed in single-cell suspension obtained from spleens. Values represent number of spots from E7-re-stimulated culture minus that from irrelevant antigen re-stimulated culture±SD.B. The absolute numbers of infiltrated CD45+CD8+ T cells were standardized per 10e6 of total tumor cells and presented as mean values±SD. *P<0.05, **P<0.01 and ***P<0.001. Similar results were obtained from two independent experiments.

FIGS. 14A-14B. Lm-LLO treatment decreases the levels of splenic and tumor infiltrating MDSC. (FIG. 14A) The percentage of splenic CD11b+Gr-1+MDSC from treated and control mice C57BL/6 mice (n=5). (FIG. 14B) The absolute numbers of infiltrated CD45+CD11b+Gr-1+MDSC standardized per 10e6 of total tumor cells are presented as mean values±SD. *P<0.05. Similar results were obtained from two independent experiments.

FIGS. 15A-15B. Lm-LLO treatment decreases the levels of splenic and tumor infiltrating Treg cells. (FIG. 15A) The percentage of CD4+FoxP3+Treg cells within CD4+ cell population of splenocytes from experimental and control groups. (FIG. 15B) The absolute numbers of infiltrated CD45+CD4+FoxP3+Treg cells standardized per 10e6 of total tumor cells are presented as mean values±SD. *P<0.05. Similar results were obtained from two independent experiments.

FIGS. 16A-16B. Lm-LLO infection upregulates PD-L1 expression on monocyte-derived human DC surface. (FIG. 16A) Fold increase of PD-L1 expression on human DC after treatment with different concentrations of Lm-LLO over non-treated control. (FIG. 16B) Representative histogram of PD-L1 expression on human DC treated with different concentrations of Lm-LLO. Similar results were obtained from three independent experiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

This invention provides in one embodiment, an immunogenic composition comprising an immune checkpoint inhibitor and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, an immunogenic composition comprising an immune checkpoint inhibitor or agonist and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is an immunogenic composition comprising a T-cell stimulator, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof. In another embodiment, administration of the T-cell stimulator may be concurrent with administration of the recombinant Listeria strain. In another embodiment, administration of the T-cell stimulator may be prior to administration of the recombinant Listeria strain. In another embodiment, administration of the T-cell stimulator may be after administration of the recombinant Listeria strain.

In another embodiment, provided herein is an immunogenic composition comprising an immune checkpoint inhibitor, a T-cell stimulator, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof. In another embodiment, administration of the checkpoint inhibitor and the T-cell stimulator may be concurrent with administration of the recombinant Listeria strain. In another embodiment, administration of the checkpoint inhibitor and T-cell stimulator may be prior to administration of the recombinant Listeria strain. In another embodiment, administration of the checkpoint inhibitor and the T-cell stimulator may be after administration of the recombinant Listeria strain.

In another embodiment, provided herein is an immunogenic composition comprising a programmed cell death receptor-1 (PD-1) signaling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is an immunogenic composition comprising a programmed cell death receptor-1 (PD-1) signaling pathway inhibitor, or a CD-80/86 and CTLA4 signalling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of eliciting an enhanced anti-tumor T cell immune response in a subject, the method comprising the step of administering to said subject an immunogenic composition comprising a programmed cell death receptor-1 (PD-1) signaling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of eliciting an enhanced anti-tumor T cell immune response in a subject, the method comprising the step of administering to said subject an immunogenic composition comprising an immune checkpoint inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of eliciting an enhanced anti-tumor T cell immune response in a subject, the method comprising the step of administering to said subject an immunogenic composition comprising a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of eliciting an enhanced anti-tumor T cell immune response in a subject, the method comprising the step of administering to said subject an immunogenic composition comprising an immune checkpoint inhibitor, a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, administration of a checkpoint inhibitor may be concurrent with administration of the recombinant Listeria strain. In another embodiment, administration of a checkpoint inhibitor may be prior to administration of the recombinant Listeria strain. In another embodiment, administration may be after administration of the recombinant Listeria strain.

In one embodiment, provided herein is a method of inhibiting tumor-mediated immunosuppression in a subject, the method comprising the step of administering to said subject an immunogenic composition as provided herein.

In another embodiment, provided herein is a method of inhibiting tumor-mediated immunosuppression in a subject, the method comprising the step of administering to said subject an immunogenic composition comprising a programmed cell death receptor-1 (PD-1) signaling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of inhibiting tumor-mediated immunosuppression in a subject, the method comprising the step of administering to said subject an immunogenic composition comprising an immune checkpoint inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of inhibiting tumor-mediated immunosuppression in a subject, the method comprising the step of administering to said subject an immunogenic composition comprising a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of inhibiting tumor-mediated immunosuppression in a subject, the method comprising the step of administering to said subject an immunogenic composition comprising an immune checkpoint inhibitor, a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of preventing or treating a tumor growth or cancer in a subject, the method comprising the step of administering to said subject an immunogenic composition comprising a programmed cell death receptor-1 (PD-1) signaling pathway inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, the heterologous antigen is a tumor-associated antigen. In another embodiment, the tumor-associated antigen is a naturally occurring tumor-associated antigen. In another embodiment, the tumor-associated antigen is a synthetic tumor-associated antigen.

In one embodiment, provided herein is a method of increasing a ratio of T effector cells to regulatory T cells (Tregs) in the spleen and tumor microenvironments of a subject, comprising administering the immunogenic composition provided herein. In another embodiment, provided herein is a method of increasing the ratio of T effector cells to regulatory T cells (Tregs) in the spleen and tumor of a subject, the method comprising the step of administering to said subject an immunogenic composition comprising an immune checkpoint inhibitor, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of increasing the ratio of T effector cells to regulatory T cells (Tregs) in the spleen and tumor of a subject, the method comprising the step of administering to said subject an immunogenic composition comprising a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, provided herein is a method of increasing the ratio of T effector cells to regulatory T cells (Tregs) in the spleen and tumor of a subject, the method comprising the step of administering to said subject an immunogenic composition comprising an immune checkpoint inhibitor, a T-cell stimulator, and a recombinant Listeria strain comprising a nucleic acid molecule, said nucleic acid molecule comprising a first open reading frame encoding fusion polypeptide, wherein said fusion polypeptide comprises a truncated Listeriolysin O protein, a truncated ActA protein, or a PEST amino acid sequence fused to a heterologous antigen or fragment thereof.

In another embodiment, increasing a ratio of T effector cells to regulatory T cells (Tregs) in the spleen and tumor microenvironments in a subject allows for a more profound anti-tumor response in said subject.

In one embodiment, the recombinant Listeria strain provided herein lacks antibiotic resistance genes. In another embodiment, the recombinant Listeria strain provided herein comprises a plasmid comprising a nucleic acid encoding an antibiotic resistance gene.

In one embodiment, the recombinant Listeria provided herein is capable of escaping the phagolysosome.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In one embodiment, the present invention provides methods of treating, protecting against, and inducing an immune response against a tumor or a cancer, comprising the step of administering to a subject the immunogenic composition provided herein.

In one embodiment, the present invention provides a method of preventing or treating a tumor or cancer in a human subject, comprising the step of administering to the subject the immunogenic composition strain provided herein comprising an immune checkpoint inhibitor, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof, whereby administration of said composition induces an immune response against the heterologous antigen, thereby treating a tumor or cancer in a human subject. In another embodiment, the heterologous antigen comprises a tumor-associated antigen, whereby the recombinant Listeria strain induces an immune response against the tumor-associated antigen, thereby treating a tumor or cancer in a human subject. In another embodiment, the immune response is an T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response.

In one embodiment, the present invention provides a method of preventing or treating a tumor or cancer in a human subject, comprising the step of administering to the subject the immunogenic composition strain provided herein comprising a T-cell stimulator, and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof, whereby administration of said composition induces an immune response against the heterologous antigen, thereby treating a tumor or cancer in a human subject. In another embodiment, the heterologous antigen comprises a tumor-associated antigen, whereby the recombinant Listeria strain induces an immune response against the tumor-associated antigen, thereby treating a tumor or cancer in a human subject. In another embodiment, the immune response is an T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response.

In one embodiment, the present invention provides a method of preventing or treating a tumor or cancer in a human subject, comprising the step of administering to the subject the immunogenic composition strain provided herein comprising an immune checkpoint inhibitor, a T-cell stimulator and a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof, whereby administration of said composition induces an immune response against the heterologous antigen, thereby treating a tumor or cancer in a human subject. In another embodiment, the heterologous antigen comprises a tumor-associated antigen, whereby the recombinant Listeria strain induces an immune response against the tumor-associated antigen, thereby treating a tumor or cancer in a human subject. In another embodiment, the immune response is an T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response.

In another embodiment, the present invention provides a method of protecting a subject against a tumor or cancer, comprising the step of administering to the subject the immunogenic composition provided herein. In another embodiment, the present invention provides a method of inducing regression of a tumor in a subject, comprising the step of administering to the subject the immunogenic composition provided herein. In another embodiment, the present invention provides a method of reducing the incidence or relapse of a tumor or cancer, comprising the step of administering to the subject the immunogenic composition provided herein. In another embodiment, the present invention provides a method of suppressing the formation of a tumor in a subject, comprising the step of administering to the subject the immunogenic composition provided herein. In another embodiment, the present invention provides a method of inducing a remission of a cancer in a subject, comprising the step of administering to the subject the immunogenic composition provided herein. In one embodiment, the nucleic acid molecule comprising a first open reading frame encoding a fusion polypeptide is integrated into the Listeria genome. In another embodiment, the nucleic acid is in a plasmid in said recombinant Listeria strain. In another embodiment, the nucleic acid molecule is in a bacterial artificial chromosome in said recombinant Listeria strain.

In one embodiment, the Listeria genome comprises a deletion of the endogenous actA gene, which in one embodiment is a virulence factor. In one embodiment, such a deletion provides a more attenuated and thus safer Listeria strain for human use. According, in one embodiment, the heterologous antigen or antigenic polypeptide is integrated in frame with LLO in the Listeria chromosome. In another embodiment, the integrated nucleic acid molecule is integrated into the actA locus. In another embodiment, the chromosomal nucleic acid encoding ActA is replaced by a nucleic acid molecule encoding an antigen.

It will be appreciated by a skilled artisan that the term, “antigenic polypeptide” refer to a polypeptide, peptide or recombinant peptide as described hereinabove that is processed and presented on MHC class I and/or class II molecules present in a subject's cells leading to the mounting of an immune response when present in, or, in another embodiment, detected by, the host.

In one embodiment, an antigen may be foreign, that is, heterologous to the host and is referred to as a “heretologous antigen” herein. In another embodiment, a heterologous antigen is heterologous to a Listeria strain provided herein that recombinantly expresses said antigen. In another embodiment, a heterologous antigen is heterologous to the host and a Listeria strain provided herein that recombinantly expresses said antigen. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen.

In one embodiment, the nucleic acid molecule provided herein comprises a first open reading frame encoding encoding a fusion polypeptide, wherein said fusion polypeptide comprises a truncated Listeriolysin O protein (LLO), a truncated ActA protein, or a PEST amino acid sequence fused to a heterologous antigen or fragment thereof. In another embodiment, the truncated LLO protein is a N-terminal LLO or fragment thereof. In another embodiment, the truncated ActA protein is a N-terminal ActA protein or fragment thereof.

In one embodiment, the nucleic acid molecule provided herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains provided herein comprise a mutation in the endogenous dal/dat genes. In another embodiment, the Listeria lacks the dal/dat genes.

In another embodiment, a nucleic acid molecule of the methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the present invention.

In another embodiment, the recombinant Listeria is attenuated. In another embodiment, the recombinant Listeria is an attenuated auxotrophic strain. In another embodiment, the recombinant Listeria is an Lm-LLO-E7 strain described in U.S. Pat. No. 8,114,414, which is incorporated by reference herein in its entirety.

In one embodiment the attenuated strain is Lm dal(−)dat(−) (Lmdd). In another embodiment, the attenuated strains is Lm dal(−)dat(−)ΔactA (LmddA). LmddA is based on a Listeria vector which is attenuated due to the deletion of virulence gene actA and retains the plasmid for a desired heterologous antigen or trunctated LLO expression in vivo and in vitro by complementation of dal gene.

In another embodiment, the attenuated strain is LmΔactA. In another embodiment, the attenuated strain is LmΔprfA. In another embodiment, the attenuated strain is LmΔPlcB. In another embodiment, the attenuated strain is LmΔplcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain used in the invention is a Listeria strain that expresses a non-hemolytic LLO.

In one embodiment, the Listeria disclosed herein is a Listeria vaccine strain. In another embodiment, the therapy disclosed herein that makes use of a Listeria strain also disclosed herein is a Listeria-based immunotherapy.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as provided herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, said metabolic enzyme is an amino acid metabolism enzyme. In another embodiment, said metabolic enzyme catalyzes a formation of an amino acid used for a cell wall synthesis in said recombinant Listeria strain. In another embodiment, said metabolic enzyme is an alanine racemase enzyme. In another embodiment, said metabolic enzyme is a D-amino acid transferase enzyme. Each possibility represents a separate embodiment of the methods and compositions as provided herein.

In one embodiment, said auxotrophic Listeria strain comprises an episomal expression vector comprising a metabolic enzyme that complements the auxotrophy of said auxotrophic Listeria strain. In another embodiment, the construct is contained in the Listeria strain in an episomal fashion. In another embodiment, the foreign antigen is expressed from a vector harbored by the recombinant Listeria strain. In another embodiment, said episomal expression vector lacks an antibiotic resistance marker. In one embodiment, an antigen of the methods and compositions as provided herein is fused to a truncated Listerolysin O protein (LLO), a truncated ActA protein or a PEST amino acid sequence. In another embodiment, an antigen of the methods and compositions as provided herein is fused to a truncated LLO. In another embodiment, an antigen of the methods and compositions as provided herein is fused to a truncated ActA protein. In another embodiment, an antigen of the methods and compositions as provided herein, is fused to a PEST amino acid sequence.

In another embodiment, the Listeria strain is deficient in an AA metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc). In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, provided herein is a nucleic acid molecule that is used to transform the Listeria in order to arrive at a recombinant Listeria. In another embodiment, the nucleic acid provided herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence in the recombinant Listeria.

In yet another embodiment the Listeria strain is an inlA mutant, an inlB mutant, an inlC mutant, an inlJ mutant, prfA mutant, ActA mutant, a dal/dat mutant, a prfA mutant, a plcB deletion mutant, or a double mutant lacking both plcA and plcB. In another embodiment, the Listeria comprise a deletion or mutation of these genes individually or in combination. In another embodiment, the Listeria provided herein lack each one of genes. In another embodiment, the Listeria provided herein lack at least one and up to ten of any gene provided herein, including the actA, prfA, and dal/dat genes. In another embodiment, the prfA mutant is a D133V prfA mutant.

In one embodiment, the live attenuated Listeria is a recombinant Listeria. In another embodiment, the recombinant Listeria comprises a mutation or a deletion of a genomic internalin C (inlC) gene. In another embodiment, the recombinant Listeria comprises a mutation or a deletion of a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a vaccine backbone. Each possibility represents a separate embodiment of the present invention.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome. Each possibility represents a separate embodiment of the present invention.

In one embodiment, the recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain provided herein comprise an inactivating mutation of the endogenous actA gene. In another embodiment, the recombinant Listeria strain provided herein comprise an inactivating mutation of the endogenous inlB gene. In another embodiment, the recombinant Listeria strain provided herein comprise an inactivating mutation of the endogenous inlC gene. In another embodiment, the recombinant Listeria strain provided herein comprise an inactivating mutation of the endogenous actA and inlB genes. In another embodiment, the recombinant Listeria strain provided herein comprise an inactivating mutation of the endogenous actA and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation in any single gene or combination of the following genes: actA, dal, dat, inlB, inlC, prfA, plcA, plcB.

It will be appreciated by the skilled artisan that the term “mutation” and grammatical equivalents thereof, include any type of mutation or modification to the sequence (nucleic acid or amino acid sequence), and includes a deletion mutation, a truncation, an inactivation, a disruption, replacement or a translocation. These types of mutations are readily known in the art.

In one embodiment, in order to select for an auxotrophic bacteria comprising a plasmid encoding a metabolic enzyme or a complementing gene provided herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.).

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with this invention.

In one embodiment, the N-terminal LLO protein fragment and heterologous antigen are, in another embodiment, fused directly to one another. In another embodiment, the genes encoding the N-terminal LLO protein fragment and heterologous antigen are fused directly to one another. In another embodiment, the N-terminal LLO protein fragment and heterologous antigen are operably attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is the N-terminal-most portion of the fusion protein. In another embodiment, a truncated LLO is truncated at the C-terminal to arrive at an N-terminal LLO. As provided herein, recombinant Listeria strains expressing LLO unexpectedly increase CD4+FoxP3− T cell and CD8+ T cell number in the spleen to a level higher than a recombinant Listeria strain not expressing truncated LLO (Example 5), thereby demonstrating that expansion of CD4+FoxP3− T cells and CD8+ T cells is directly mediated by LLO (Example 6). As further provided herein, the recombinant Listeria episomally expressing a truncated LLO unexpectedly increases the ratio of CD4+FoxP3− T cell and CD8+ T cell to CD4+FoxP3+ T cell (regulatory T cells or Tregs) by inducing the expansion of CD4+FoxP3− T cell and CD8+T, without reducing the number to Tregs, thereby decreasing the frequency of Tregs in a proportionate manner.

In one embodiment, the truncated ActA protein and heterologous antigen are, in another embodiment, fused directly to one another. In another embodiment, the genes encoding the truncated ActA protein and heterologous antigen are fused directly to one another. In another embodiment, the truncated ActA protein and heterologous antigen are operably attached via a linker peptide. In another embodiment, the truncated ActA protein and heterologous antigen are attached via a heterologous peptide. In another embodiment, the truncated ActA protein is N-terminal to the heterologous antigen. In another embodiment, the truncated ActA protein is the N-terminal-most portion of the fusion protein. In another embodiment, a truncated ActA protein is truncated at the C-terminal to arrive at an N-terminal ActA.

In one embodiment, the PEST amino acid sequence and heterologous antigen are, in another embodiment, fused directly to one another. In another embodiment, the genes encoding the PEST amino acid sequence and heterologous antigen are fused directly to one another. In another embodiment, the PEST amino acid sequence and heterologous antigen are operably attached via a linker peptide. In another embodiment, the PEST amino acid sequence and heterologous antigen are attached via a heterologous peptide. In another embodiment, the PEST amino acid sequence is N-terminal to the heterologous antigen.

In one embodiment, a nucleic acid molecule comprised in a Listeria of this invention encodes a recombinant polypeptide. In another embodiment, the recombinant Listeria strain provided herein expresses the recombinant polypeptide. In another embodiment, the recombinant Listeria strain comprises a plasmid that encodes the recombinant polypeptide. In another embodiment, a recombinant nucleic acid provided herein is in a plasmid in the recombinant Listeria strain provided herein. In another embodiment, the plasmid is an episomal plasmid that does not integrate into said recombinant Listeria strain's chromosome. In another embodiment, the plasmid is an integrative plasmid that integrates into said Listeria strain's chromosome. In another embodiment, the plasmid is a multicopy plasmid.

In one embodiment, the method comprises the step of co-administering the recombinant Listeria with an additional therapy. In another embodiment, the additional therapy is surgery, chemotherapy, an immunotherapy or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the antibody therapy is an anti-PD1, anti-CTLA4. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in U.S. Pat. No. 6,991,785, incorporated by reference herein.

In one embodiment, the heterologous antigen is a tumor-associated antigen. In one embodiment, the recombinant Listeria strain of the compositions and methods as provided herein express a fusion polypeptide comprising an antigen that is expressed by a tumor cell.

In another embodiment, the tumor-associated antigen is a human papilloma virus (HPV). In another embodiment, the tumor-associated antigen is HPV-E7. In another embodiment, the antigen is HPV-E6. In another embodiment, the antigen is Her-2. In another embodiment, the antigen is NY-ESO-1. In another embodiment, the antigen is telomerase. In another embodiment, the antigen is SCCE. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is selected from E7, E6, Her-2, NY-ESO-1, telomerase, SCCE, WT-1, HIV-1 Gag, Proteinase 3, Tyrosinase related protein 2. In another embodiment, the antigen is a tumor-associated antigen. In another embodiment, the antigen is an infectious disease antigen.

In another embodiment, the tumor-associated antigen is an angiogenic antigen. In another embodiment, the angiogenic antigen is expressed on both activated pericytes and pericytes in tumor angiogenic vasculature, which in another embodiment, is associated with neovascularization in vivo. In another embodiment, the angiogenic antigen is HMW-MAA. In another embodiment, the angiogenic antigen is one known in the art and are provided in WO2010/102140, which is incorporated by reference herein.

In one embodiment, compositions of the present invention induce a strong innate stimulation of interferon-gamma, which in one embodiment, has anti-angiogenic properties. In one embodiment, a Listeria of the present invention induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J Immunol. 2001 Feb. 15; 166(4):2276-82, incorporated herein by reference in its entirety). In another embodiment, methods of the present invention increase a level of interferon-gamma producing cells. In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, compositions of the present invention induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, mesothelin, EGFRVIII.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment, the heterologous antigen provided herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma or. In another embodiment, the antigen for the compositions and methods as provided herein are melanoma-associated antigens, which in one embodiment are TRP-2, MAGE-1, MAGE-3, gp-100, tyrosinase, HSP-70, beta-HCG, or a combination thereof.

In one embodiment, the antigen is a chimeric Her2 antigen described in U.S. Pat. No. 9,084,747, which is hereby incorporated by reference herein in its entirety.

In another embodiment, the antigen is HPV-E7. In another embodiment, the antigen is HPV-E6. In another embodiment, the antigen is Her-2/neu. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is p53. In another embodiment, the antigen is carboxic anhydrase IX (CAIX). In another embodiment, the antigen is PSMA. In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is selected from HPV-E7, HPV-E6, Her-2, NY-ESO-1, telomerase (TERT), SCCE, HMW-MAA, EGFR-III, survivin, baculoviral inhibitor of apoptosis repeat-containing 5 (BIRC5), WT-1, HIV-1 Gag, CEA, LMP-1, p53, PSMA, PSCA, Proteinase 3, Tyrosinase related protein 2, Muc1, or a combination thereof.

In one embodiment, a fusion polypeptide expressed by the Listeria of the present invention may comprise a neuropeptide growth factor antagonist, which in one embodiment is [D-Arg1, D-Phe5, D-Trp7,9, Leu11] substance P, [Arg6, D-Trp7,9, NmePhe8]substance P(6-11). These and related embodiments embodiments are understood by one of skill in the art.

In another embodiment, the heterologous antigen is an infectious disease antigen. In one embodiment, the antigen is an auto antigen or a self-antigen.

In another embodiment, the heterologous antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In another embodiments, the heterologous antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis. Each antigen represents a separate embodiment of the methods and compositions as provided herein.

The immune response induced by methods and compositions as provided herein is, in another embodiment, a T cell response. In another embodiment, the immune response comprises a T cell response. In another embodiment, the response is a CD8⁺ T cell response. In another embodiment, the response comprises a CD8⁺ T cell response.

In one embodiment, a recombinant Listeria of the compositions and methods as provided herein comprise a nucleic acid encoding an angiogenic polypeptide or angiogenic antigen. In another embodiment, anti-angiogenic approaches to cancer therapy are very promising, and in one embodiment, one type of such anti-angiogenic therapy targets pericytes. In another embodiment, molecular targets on vascular endothelial cells and pericytes are important targets for antitumor therapies. In another embodiment, the platelet-derived growth factor receptor (PDGF-B/PDGFR-β) signaling is important to recruit pericytes to newly formed blood vessels. Thus, in one embodiment, angiogenic polypeptides as provided herein inhibit molecules involved in pericyte signaling, which in one embodiment, is PDGFR-β.

In one embodiment, the compositions of the present invention comprise an angiogenic factor, or an immunogenic fragment thereof, where in one embodiment, the immunogenic fragment comprises one or more epitopes recognized by the host immune system. In one embodiment, an angiogenic factor is a molecule involved in the formation of new blood vessels. In one embodiment, the angiogenic factor is VEGFR2. In another embodiment, an angiogenic factor of the present invention is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; survivin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-β), endoglin and TGF-β receptors; monocyte chemotactic protein-1 (MCP-1); Integrins αVβ3, αVβ5 and α5β1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods of the present invention is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGFbeta co-receptor.

In one embodiment, the immunogenic compositions provided herein are useful for preventing, suppressing, inhibiting, or treating an autoimmune disease. In one embodiment, the autoimmune disease is any autoimmune disease known in the art, including, but not limited to, a rheumatoid arthritis (RA), insulin dependent diabetes mellitus (Type 1 diabetes), multiple sclerosis (MS), Crohn's disease, systemic lupus erythematosus (SLE), scleroderma, Sjogren's syndrome, pemphigus vulgaris, pemphigoid, addison's disease, ankylosing spondylitis, aplastic anemia, autoimmune hemolytic anemia, autoimmune hepatitis, coeliac disease, dermatomyositis, Goodpasture's syndrome, Graves' disease, Guillain-Barre syndrome, Hashimoto's disease, idiopathic leucopenia, idiopathic thrombocytopenic purpura, male infertility, mixed connective tissue disease, myasthenia gravis, pernicious anemia, phacogenic uveitis, primary biliary cirrhosis, primary myxoedema, Reiter's syndrome, stiff man syndrome, thyrotoxicosis, ulceritive colitis, and Wegener's granulomatosis. In another embodiment, the invention is also drawn to the agonist antibody directed against ICOS according to the invention or a derivative thereof for use for treating an inflammatory disorder selected in the group consisting of inflammatory disorder of the nervous system such as multiple sclerosis, mucosal inflammatory disease such as inflammatory bowel disease, asthma or tonsillitis, inflammatory skin disease such as dermatitis, psoriasis or contact hypersensitivity, and autoimmune arthritis such as rheumatoid arthritis.

In one embodiment, compositions and methods of use thereof as provided herein generate effector T cells that are able to infiltrate the tumor, destroy tumor cells and eradicate the disease. In another embodiment, methods of use of this invention increase more infiltration by T effector cells. In another embodiment, T effector cells comprise CD45+CD8+ T cells. In another embodiment, T effector cells comprise CD4+Fox3P T cells.

In one embodiment, naturally occurring tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as provided herein increase TILs, up-regulate adhesion molecules (in one embodiment, ICAM-1, V-CAM-1, VAP-1, E-selectin, or a combination thereof), up-regulate pro-inflammatory cytokines (in one embodiment, IFN-γ, TNF-α, IL-1, or a combination thereof), or a combination thereof.

In one embodiment the HPV antigen is an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type. Each possibility represents a separate embodiment of the present invention.

In one embodiment, the HPV E6 is from HPV-16. In another embodiment, the HPV E7 is from HPV-16. In another embodiment, the HPV-E6 is from HPV-18. In another embodiment, the HPV-E7 is from HPV-18. In another embodiment, an HPV E6 antigen is utilized instead of or in addition to an E7 antigen in a composition or method of the present invention for treating or ameliorating an HPV-mediated disease, disorder, or symptom. In another embodiment, an HPV-16 E6 and E7 is utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria provided herein. In another embodiment, the HPV-16 E6 and E7 antigens and the HPV-18 E6 and E7 antigens are expressed from the chromosome of a recombinant Listeria provided herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In one embodiment, the disease provided herein is a cancer or a tumor. In one embodiment, the cancer treated by a method of the present invention is breast cancer. In another embodiment, the cancer is a cervical cancer. In another embodiment, the cancer is an Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. In another embodiment, the cancer is mesothelioma. Each possibility represents a separate embodiment of the present invention.

In one embodiment, the truncated LLO comprises a PEST amino acid (AA) sequence. In another embodiment, the PEST amino acid sequence is KENSISSMAPPASPPASPKTPIEKKHADEIDK (SEQ ID NO: 1). In another embodiment, fusion of an antigen to other LM PEST AA sequences from Listeria will also enhance immunogenicity of the antigen.

The N-terminal LLO protein fragment of methods and compositions of the present invention comprises, in another embodiment, SEQ ID No: 2. In another embodiment, the fragment comprises an LLO signal peptide. In another embodiment, the fragment comprises SEQ ID No: 3. In another embodiment, the fragment consists approximately of SEQ ID No: 3. In another embodiment, the fragment consists essentially of SEQ ID No: 3. In another embodiment, the fragment corresponds to SEQ ID No: 3. In another embodiment, the fragment is homologous to SEQ ID No: 3. In another embodiment, the fragment is homologous to a fragment of SEQ ID No: 3. The ALLO used in some of the Examples was 416 AA long (exclusive of the signal sequence), as 88 residues from the amino terminus which is inclusive of the activation domain containing cysteine 484 were truncated. It will be clear to those skilled in the art that any ALLO without the activation domain, and in particular without cysteine 484, are suitable for methods and compositions of the present invention. In another embodiment, fusion of a heterologous antigen to any ALLO, including the PEST AA sequence, SEQ ID NO: 1, enhances cell mediated and anti-tumor immunity of the antigen. Each possibility represents a separate embodiment of the present invention.

The LLO protein utilized to construct vaccines of the present invention has, in another embodiment, the sequence: MKKIMLVFITLILVSLPIAQQTEAKDASAFNKENSISSMAPPASPPASPKTPIEKKHADE IDKYIQGLDYNKNNVLVYHGDAVTNVPPRKGYKDGNEYIVVEKKKKSINQNNADIQ VVNAISSLTYPGALVKANSELVENQPDVLPVKRDSLTLSIDLPGMTNQDNKIVVKNA TKSNVNNAVNTLVERWNEKYAQAYPNVSAKIDYDDEMAYSESQLIAKFGTAFKAV NNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTDGKINIDHSGGYVAQFNISWDEVNYDPEGNEIVQHKNWSENNK SKLAHFTSSIYLPGNARNINVYAKECTGLAWEWWRTVIDDRNLPLVKNRNISIWGTT LYPKYSNKVDNPIE (GenBank Accession No. P13128; SEQ ID NO: 4; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention.

In another embodiment, the N-terminal fragment of an LLO protein utilized in compositions and methods of the present invention has the sequence:

(SEQ ID NO: 2)
MKKIMLVFITLILVSLPIAQQTEAKDASAFNKENSISSVAPPASPP
ASPKTPIEKKHADEIDKYIQGLDYNKNNVLVYHGDAVTNVPPRKGY
KDGNEYIVVEKKKKSINQNNADIQVVNAISSLTYPGALVKANSELV
ENQPDVLPVKRDSLTLSIDLPGMTNQDNKIVVKNATKSNVNNAVNT
LVERWNEKYAQAYSNVSAKIDYDDEMAYSESQLIAKFGTAFKAVNN
SLNVNFGAISEGKMQEEVISFKQIYYNVNVNEPTRPSRFFGKAVTK
EQLQALGVNAENPPAYISSVAYGRQVYLKLSTNSHSTKVKAAFDAA
VSGKSVSGDVELTNIIKNSSFKAVIYGGSAKDEVQIIDGNLGDLRD
ILKKGATFNRETPGVPIAYTTNFLKDNELAVIKNNSEYIETTSKAY
TDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 3)
MKKIMLVFITLILVSLPIAQQTEAKDASAFNKENSISSVAPPASPP
ASPKTPIEKKHADEIDKYIQGLDYNKNNVLVYHGDAVTNVPPRKGY
KDGNEYIVVEKKKKSINQNNADIQVVNAISSLTYPGALVKANSELV
ENQPDVLPVKRDSLTLSIDLPGMTNQDNKIVVKNATKSNVNNAVNT
LVERWNEKYAQAYSNVSAKIDYDDEMAYSESQLIAKFGTAFKAVNN
SLNVNFGAISEGKMQEEVISFKQIYYNVNVNEPTRPSRFFGKAVTK
EQLQALGVNAENPPAYISSVAYGRQVYLKLSTNSHSTKVKAAFDAA
VSGKSVSGDVELTNIIKNSSFKAVIYGGSAKDEVQIIDGNLGDLRD
ILKKGATFNRETPGVPIAYTTNFLKDNELAVIKNNSEYIETTSKAY
TD.

In another embodiment, “truncated LLO” or “ALLO” refers to a fragment of LLO that comprises a putative PEST amino acid sequence. In another embodiment, the terms refer to an LLO fragment that comprises a putative PEST domain. In another embodiment, their terms “truncated LLO” and “N-terminal LLO” are used interchangeably herein.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location. In another embodiment, the LLO is rendered non-hemolytic by a deletion or mutation of the cholesterol binding domain (CBD) as detailed in U.S. Pat. No. 8,771,702, which is incorporated by reference herein.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

In another embodiment, a homologous LLO refers to identity to an LLO sequence (e.g. to one of SEQ ID No: 2-4) of greater than 70%. In another embodiment, a homologous LLO refers to identity to one of SEQ ID No: 2-4 of greater than 72%. In another embodiment, a homologous refers to identity to one of SEQ ID No: 2-4 of greater than 75%. In another embodiment, a homologous refers to identity to one of SEQ ID No: 2-4 of greater than 78%. In another embodiment, a homologous refers to identity to one of SEQ ID No: 2-4 of greater than 80%. In another embodiment, a homologous refers to identity to one of SEQ ID No: 2-4 of greater than 82%. In another embodiment, a homologous refers to identity to one of SEQ ID No: 2-4 of greater than 83%. In another embodiment, a homologous refers to identity to one of SEQ ID No: 2-4 of greater than 85%. In another embodiment, a homologous refers to identity to one of SEQ ID No: 2-4 of greater than 87%. In another embodiment, a homologous refers to identity to one of SEQ ID No: 2-4 of greater than 88%. In another embodiment, a homologous refers to identity to one of SEQ ID No: 2-4 of greater than 90%. In another embodiment, a homologous refers to identity to one of SEQ ID No: 2-4 of greater than 92%. In another embodiment, a homologous refers to identity to one of SEQ ID No: 2-4 of greater than 93%. In another embodiment, a homologous refers to identity to one of SEQ ID No: 2-4 of greater than 95%. In another embodiment, a homologous refers to identity to one of SEQ ID No: 2-4 of greater than 96%. In another embodiment, a homologous refers to identity to one of SEQ ID No: 2-4 of greater than 97%. In another embodiment, a homologous refers to identity to one of SEQ ID No: 2-4 of greater than 98%. In another embodiment, a homologous refers to identity to one of SEQ ID No: 2-4 of greater than 99%. In another embodiment, a homologous refers to identity to one of SEQ ID No: 2-4 of 100%.

In another embodiment, the term “homology,” when in reference to any nucleic acid sequence provided herein similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from the sequences provided herein of greater than 68%. In another embodiment, “homology” refers to identity to a sequence selected from the sequences provided herein of greater than 70%. In another embodiment, “homology” refers to identity to a sequence selected from the sequences provided herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5×Denhardt's solution, 10% dextran sulfate, and 20 g/ml denatured, sheared salmon sperm DNA.

In another embodiment of the methods and compositions as provided herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment as provided herein.

In one embodiment, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), *-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides as provided herein may also include one or more modified amino acids or one or more non-amino acid monomers (e.g. fatty acids, complex carbohydrates etc).

In one embodiment, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In one embodiment, the Listeria strain provided herein encodes a fusion protein of truncated LLO fused to an HPV-E7 antigen. In another embodiment, a sequence encoding a tLLO-E7 fusion protein comprises SEQ ID NO: 13: atgaaaaaaataatgctagtttttattacacttatattagttagtctaccaattgcgcaacaaactgaagcaaaggatgcatctgcattcaata aagaaaattcaatttcatccatggcaccaccagcatctccgcctgcaagtcctaagacgccaatcgaaaagaaacacgcggatgaaat cgataagtatatacaaggattggattacaataaaaacaatgtattagtataccacggagatgcagtgacaaatgtgccgccaagaaaag gttacaaagatggaaatgaatatattgttgtggagaaaaagaagaaatccatcaatcaaaataatgcagacattcaagttgtgaatgcaat ttcgagcctaacctatccaggtgctctcgtaaaagcgaattcggaattagtagaaaatcaaccagatgttctccctgtaaaacgtgattcat taacactcagcattgatttgccaggtatgactaatcaagacaataaaatagttgtaaaaaatgccactaaatcaaacgttaacaacgcagt aaatacattagtggaaagatggaatgaaaaatatgctcaagcttatccaaatgtaagtgcaaaaattgattatgatgacgaaatggcttac agtgaatcacaattaattgcgaaatttggtacagcatttaaagctgtaaataatagcttgaatgtaaacttcggcgcaatcagtgaaggga aaatgcaagaagaagtcattagttttaaacaaatttactataacgtgaatgttaatgaacctacaagaccttccagatttttcggcaaagctg ttactaaagagcagttgcaagcgcttggagtgaatgcagaaaatcctcctgcatatatctcaagtgtggcgtatggccgtcaagtttatttg aaattatcaactaattcccatagtactaaagtaaaagctgcttttgatgctgccgtaagcggaaaatctgtctcaggtgatgtagaactaac aaatatcatcaaaaattcttccttcaaagccgtaatttacggaggttccgcaaaagatgaagttcaaatcatcgacggcaacctcggaga cttacgcgatattttgaaaaaaggcgctacttttaatcgagaaacaccaggagttcccattgcttatacaacaaacttcctaaaagacaatg aattagctgttattaaaaacaactcagaatatattgaaacaacttcaaaagcttatacagatggaaaaattaacatcgatcactctggagga tacgttgctcaattcaacatttcttgggatgaagtaaattatgatctcgagCATGGAGATACACCTACATTGCATG AATATATGTTAGATTTGCAACCAGAGACAACTGATCTCTACTGTTATGAGCAATT AAATGACAGCTCAGAGGAGGAGGATGAAATAGATGGTCCAGCTGGACAAGCAGA ACCGGACAGAGCCCATTACAATATTGTAACCTTTTGTTGCAAGTGTGACTCTACG CTTCGGTTGTGCGTACAAAGCACACACGTAGACATTCGTACTTTGGAAGACCTGT TAATGGGCACACTAGGAATTGTGTGCCCCATCTGTTCTCAGAAACCA (SEQ ID NO: 13), wherein the UPPERCASE sequences encode E7, the lowercase sequences encode tLLO, and the underlined “ctcgag” sequence represents the Xho I restriction site used to ligate the tumor antigen to truncated LLO in the plasmid.

In another embodiment, an amino acid sequence encoding a tLLO fused to E7 comprises SEQ ID NO: 14:

(SEQ ID NO: 14)
MKKIMLVFITLILVSLPIAQQTEAKDASAFNKENSISSMAPPASPP
ASPKTPIEKKHADEIDKYIQGLDYNKNNVLVYHGDAVTNVPPRKGY
KDGNEYIVVEKKKKSINQNNADIQVVNAISSLTYPGALVKANSELV
ENQPDVLPVKRDSLTLSIDLPGMTNQDNKIVVKNATKSNVNNAVNT
LVERWNEKYAQAYPNVSAKIDYDDEMAYSESQLIAKFGTAFKAVNN
SLNVNFGAISEGKMQEEVISFKQIYYNVNVWEPTRPSRFFGKAVTK
EQLQALGVNAENPPAYISSVAYGRQVYLKLSTNSHSTKVKAAFDAA
VSGKSVSGDVELTNIIKNSSFKAVIYGGSAKDEVQIIDGNLGDLRD
ILKKGATFNRETPGVPIAYTTNFLKDNELAVIKNNSEYIETTSKAY
TDGKINIDHSGGYVAQFNISWDEVNYDLEHGDTPTLHEYMLDLQPE
TTDLYCYEQLMDSSEEEDEIDGPAGQAEFDRAHYNIVTFCCKCDST
LRLCVQSTHVDIRTLEDLLMGTLGIVCPICSQKP.

In one embodiment, a recombinant Listeria comprising a nucleic acid encoding a tLLO fused to E7 comprising SEQ ID NO: 14 is referred to as ADXS-HPV. In another embodiment, “ADXS-HPV” and “ADXS11-001” are used interchangeably herein.

In another embodiment, the construct or nucleic acid molecule provided herein is integrated into the Listerial chromosome using homologous recombination. Techniques for homologous recombination are well known in the art, and are described, for example, in Baloglu S, Boyle S M, et al. (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present invention further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain can be complemented.

In one embodiment of the methods and compositions as provided herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions as provided herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” as used herein describes an item that has developed or originated within the reference organism or arisen from causes within the reference organism. In another embodiment, endogenous refers to native.

It will be appreciated by the skilled artisan that the term “PEST amino acid sequence” or “PEST sequence-containing polypeptide” or “PEST sequence-containing protein” or “PEST-sequence containing peptide” may be used interchangeably have all the same meanings and qualities, and may encompass a truncated LLO protein, which in one embodiment is a N-terminal LLO, and a truncated ActA protein, which in one embodiment is an N-terminal ctA, or fragments thereof. PEST amino acid sequences are known in the art and are described in U.S. Pat. No. 7,635,479, and in US Patent Publication Serial No. 2014/0186387, both of which are hereby incorporated in their entirety herein.

In another embodiment, a PEST amino acid sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected to include, but are not limited to, other Listeria species. For example, the L. monocytogenes protein ActA contains four such sequences. These are KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: 7), and RGGIPTSEEFSSLNSGDFTDDENSETTEEEIDR (SEQ ID NO: 8). Also Streptolysin O from Streptococcus sp. contain a PEST sequence. For example, Streptococcus pyogenes Streptolysin O comprises the PEST sequence KQNTASTETTTTNEQPK (SEQ ID NO: 9) at amino acids 35-51 and Streptococcus equisimilis Streptolysin O comprises the PEST-like sequence KQNTANTETTTTNEQPK (SEQ ID NO: 10) at amino acids 38-54. Further, it is believed that the PEST sequence can be embedded within the antigenic protein. Thus, for purposes of the present invention, by “fusion” when in relation to PEST sequence fusions, it is meant that the antigenic protein comprises both the antigen and the PEST amino acid sequence either linked at one end of the antigen or embedded within the antigen.

In another embodiment, the construct or nucleic acid molecule is expressed from an episomal or plasmid vector, with a nucleic acid sequence encoding fusion polypeptide comprising a PEST amino acid sequence fused to a heterologous antigen or fragment thereof. In another embodiment, the plasmid is stably maintained in the recombinant Listeria vaccine strain in the absence of antibiotic selection. In another embodiment, the plasmid does not confer antibiotic resistance upon the recombinant Listeria. In another embodiment, the fragment is a functional fragment. In another embodiment, the fragment is an immunogenic fragment.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In another embodiment, a recombinant Listeria strain of the methods and compositions as provided herein comprise a nucleic acid molecule operably integrated into the Listeria genome as an open reading frame with an endogenous ActA sequence. In another embodiment, a recombinant Listeria strain of the methods and compositions as provided herein comprise an episomal expression vector comprising a nucleic acid molecule encoding fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387.

In another embodiment, a “functional fragment” is an immunogenic fragment and elicits an immune response when administered to a subject alone or in a vaccine composition provided herein. In another embodiment, a functional fragment has biological activity as will be understood by a skilled artisan and as further provided herein.

In another embodiment, the dose of the immune checkpoint inhibitor (e.g., a PD-1 signaling pathway inhibitor) present in the immunogenic composition that is administered to a subject is 5-10 mg/kg every 2 weeks, 5-10 mg/kg every 3 weeks, or 1-2 mg/kg every 3 weeks. In another embodiment, the dose ranges from 1-10 mg/kg every week. In another embodiment, the dose ranges from 1-10 mg/kg every 2 weeks. In another embodiment, the dose ranges from 1-10 mg/kg every 3 weeks. In another embodiment, the dose ranges from 1-10 mg/kg every 4 weeks.

In another embodiment, the dose of the recombinant Listeria strain comprised by the immunogenic composition provided herein is administered to a subject at a dose of 1×10⁷-3.31×10¹⁰ CFU. In another embodiment, the dose is 1×10⁸-3.31×10¹⁰ CFU. In another embodiment, the dose is 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁷ organisms. In another embodiment, the dose is 1×10⁸ organisms. In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

It will be appreciated by the skilled artisan that the term “Boosting” may encompass administering an additional vaccine or immunogenic composition or recombinant Listeria strain dose or immune checkpoint inhibitor alone or in combination to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In another embodiment, a method of present invention further comprises the step of boosting the subject with a recombinant Listeria strain or immune checkpoint inhibitor as provided herein. In another embodiment, the recombinant Listeria strain used in the booster inoculation is the same as the strain used in the initial “priming” inoculation. In another embodiment, the booster strain is different from the priming strain. In another embodiment, the recombinant immune checkpoint inhibitor used in the booster inoculation is the same as the inhibitor used in the initial “priming” inoculation. In another embodiment, the booster inhibitor is different from the priming inhibitor. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

In another embodiment, a method of the present invention further comprises boosting the subject with a immunogenic composition comprising a PD-1 signal pathway inhibitor and recombinant Listeria strain provided herein. In another embodiment, a method of the present invention comprises the step of administering a booster dose of the immunogenic composition comprising the recombinant Listeria strain provided herein. In another embodiment, a method of the present invention further comprises boosting the subject with a immunogenic composition comprising a T-cell stimulator and recombinant Listeria strain provided herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the recombinant Listeria provided herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Vaccine 20:1226-31 (2002); Tanghe, A., Infect. Immun. 69:3041-7 (2001). Providing antigen in different forms in the prime and the boost injections appears to maximize the immune response to the antigen. DNA vaccine priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Vaccine 20:1039-45 (2002); Billaut-Mulot, O. et al., Vaccine 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

It will be appreciated by a skilled artisan that the term “fusion polypeptide” of the methods and composition of the present invention, may in certain embodiments, be used interchangable with “recombinant polypeptide”. In another embodiment, the fusion polypeptide of methods of the present invention is expressed by the recombinant Listeria strain. In another embodiment, the expression is mediated by a nucleotide molecule carried by the recombinant Listeria strain.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art.

In one embodiment, provided herein is an immunogenic composition comprising an immune checkpoint inhibitor provided herein, a T cell stimulator provided herein, and a recombinant attenuated Listeria provided herein. In another embodiment, each component of the immunogenic compositions provided herein is administered prior to, concurrently with, of after another component of the immunogenic compositions provided herein,

In another embodiment, provided herein is an immunogenic composition comprising an immune checkpoint inhibitor and a recombinant attenuated Listeria provided herein. In another embodiment, provided herein is an immunogenic composition comprising an immune checkpoint inhibitor, a T-cell stimulator, and a recombinant attenuated Listeria provided herein. In another embodiment, In one embodiment, the immune checkpoint protein inhibitor is a Programmed Death 1 (PD-1) signaling pathway inhibitor. In another embodiment, the PD-1 signaling pathway inhibitor is a molecule blocking PD-1 receptor interactions with PD-1 Ligand 1 (PD-L1) and PD-1 Ligand 2 (PD-L2). In another embodiment, PD-L1 is also known as CD274 or B7-H1. In another embodiment, PD-L2 is also known as CD273 or B7-DC. In another embodiment, the molecule blocking PD-1 receptor interactions with PD-1 Ligand 1 (PD-L1) and PD-1 Ligand 2 (PD-L2) is a molecule interacting with PD-1, PD-L1 or PD-L2. In another embodiment, the molecule blocking PD-1 receptor interactions with PD-1 Ligand 1 (PD-L1) or PD-1 Ligand 2 (PD-L2) is a molecule interacting with PD-1, PD-L1 or PD-L2. The term “interacts” or grammatical equivalents thereof may encompass binding, or coming into contact with another molecule. In another embodiment, the molecule binds to PD-1 In another embodiment, the PD-1 signaling pathway inhibitor is an anti-PD1 antibody. In another embodiment, molecule interacting with PD-L2 is an anti-PD-L1 antibody, or a small molecule that binds PD-L1. In another embodiment, the anti-PD-L1 antibody is MEDI4736. In another embodiment, molecule interacting with PD-L2 is an anti-PD-L2 antibody, or a small molecule that binds PD-L2.

In one embodiment, the molecule that interacts with PD-1 is a truncated PD-L1 protein. In another embodiment, the truncated PD-L1 protein comprises the cytoplasmic domain of PD-L1 protein. In another embodiment, the molecule interacting with PD-1 is a truncated PD-L2 protein. In another embodiment, the truncated PD-L2 protein comprises the cytoplasmic domain of PD-L2 protein. In another embodiment, the molecule blocking PD-1 receptor interactions with PD-1 Ligand 1 (PD-L1) and PD-1 Ligand 2 (PD-L2) is a molecule interacting with PD-L1 and PD-L2. In another embodiment, the molecule interacting with PD-L1 or PD-L2 is a truncated PD-1 protein, a PD-1 mimic or a small molecule that binds PD-L1 or PD-L2. In another embodiment, the truncated PD-1 protein comprises the cytoplasmic domain of the PD-1 protein.

In one embodiment, the immune checkpoint inhibitor is a CD80/86 signaling pathway inhibitor. In another embodiment, CD80 is also known as B7.1. In another embodiment, CD86 is also known as B7.2. In another embodiment, the CD80 signaling pathway inhibitor is a small molecule that interacts with CD80. In another embodiment, the CD80 inhibitor is an anti-CD80 antibody. In another embodiment, the CD86 signaling pathway inhibitor is a small molecule that interacts with CD86. In another embodiment, the CD86 inhibitor is an anti-CD86 antibody.

In one embodiment, the immune checkpoint inhibitor is a CTLA-4 signaling pathway inhibitor. In another embodiment, CTLA-4 is also known as CD152. In another embodiment, the CTLA-4 signaling pathway inhibitor is a small molecule that interacts with CTLA-4. In another embodiment, the CTLA-4 inhibitor is an anti-CTLA-4 antibody. In another embodiment, the immune checkpoint inhibitor is a CD40 signaling pathway inhibitor. In another embodiment, the immune checkpoint inhibitor is any other antigen-presenting cell:Tcell signaling pathway inhibitor known in the art.

It will be appreciated by the skilled artisan that any immune checkpoint protein known may be any checkpoint inhibitor known in the art. An immune checkpoint protein may be selected from, but is not limited to the following: programmed cell death protein 1 (PD1), T cell membrane protein 3 (TIM3), adenosine A2a receptor (A2aR) and lymphocyte activation gene 3 (LAG3), killer immunoglobulin receptor (KIR) or cytotoxic T-lymphocyte antigen-4 (CTLA-4). In another embodiment, the checkpoint inhibitor protein is one belonging to the B7/CD28 receptor superfamily. In one embodiment, the T cell stimulator is an an antigen presenting cell (APC)/T cell agonist. In another embodiment, the T cell stimulator is a CD134 or a ligand thereof or a fragment thereof, a CD-137 or a ligand thereof or a fragment thereof, or an Includible T cell costimulator (ICOS) or a ligand thereof or a fragment thereof.

In another embodiment, provided herein is an immunogenic composition comprising a T-cell stimulator, and a recombinant attenuated Listeria provided herein. In one embodiment, the T cell stimulator is an an antigen presenting cell (APC)/T cell agonist. In another embodiment, the T cell stimulator is a CD134 or a ligand thereof or a fragment thereof, a CD-137 or a ligand thereof or a fragment thereof, or an Includible T cell costimulator (ICOS) or a ligand thereof or a fragment thereof.

In another embodiment, a composition of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In one embodiment, the method provided herein further comprises the step of co-administering with, prior to or following the administration of said recombinant Listeria strain an an immune checkpoint protein inhibitor.

In one embodiment, the immune checkpoint protein inhibitor is a Programmed Death 1 (PD-1) signaling pathway inhibitor. In another embodiment, the PD-1 signaling pathway inhibitor is a molecule blocking PD-1 receptor interactions with PD-1 Ligand 1 (PD-L1) and PD-1 Ligand 2 (PD-L2). In another embodiment, PD-L1 is also known as CD274 or B7-H1. In another embodiment, PD-L2 is also known as CD273 or B7-DC. In another embodiment, the molecule blocking PD-1 receptor interactions with PD-1 Ligand 1 (PD-L1) and PD-1 Ligand 2 (PD-L2) is a molecule interacting with PD-1, PD-L1 or PD-L2. In another embodiment, the molecule blocking PD-1 receptor interactions with PD-1 Ligand 1 (PD-L1) or PD-1 Ligand 2 (PD-L2) is a molecule interacting with PD-1, PD-L1 or PD-L2. The term “interacts” or grammatical equivalents thereof may encompass binding, or coming into contact with another molecule. In another embodiment, the molecule binds to PD-1 In another embodiment, the PD-1 signaling pathway inhibitor is an anti-PD1 antibody. In another embodiment, molecule interacting with PD-L2 is an anti-PD-L1 antibody, or a small molecule that binds PD-L1. In another embodiment, the anti-PD-L1 antibody is MEDI4736. In another embodiment, molecule interacting with PD-L2 is an anti-PD-L2 antibody, or a small molecule that binds PD-L2.

In one embodiment, the molecule that interacts with PD-1 is a truncated PD-L1 protein. In another embodiment, the truncated PD-L1 protein comprises the cytoplasmic domain of PD-L1 protein. In another embodiment, the molecule interacting with PD-1 is a truncated PD-L2 protein. In another embodiment, the truncated PD-L2 protein comprises the cytoplasmic domain of PD-L2 protein. In another embodiment, the molecule blocking PD-1 receptor interactions with PD-1 Ligand 1 (PD-L1) and PD-1 Ligand 2 (PD-L2) is a molecule interacting with PD-L1 and PD-L2. In another embodiment, the molecule interacting with PD-L1 or PD-L2 is a truncated PD-1 protein, a PD-1 mimic or a small molecule that binds PD-L1 or PD-L2. In another embodiment, the truncated PD-1 protein comprises the cytoplasmic domain of the PD-1 protein.

In one embodiment, the immune checkpoint inhibitor is a CD80/86 signaling pathway inhibitor. In another embodiment, CD80 is also known as B7.1. In another embodiment, CD86 is also known as B7.2. In another embodiment, the CD80 signaling pathway inhibitor is a small molecule that interacts with CD80. In another embodiment, the CD80 inhibitor is an anti-CD80 antibody. In another embodiment, the CD86 signaling pathway inhibitor is a small molecule that interacts with CD86. In another embodiment, the CD86 inhibitor is an anti-CD86 antibody.

In one embodiment, the immune checkpoint inhibitor is a CTLA-4 signaling pathway inhibitor. In another embodiment, CTLA-4 is also known as CD152. In another embodiment, the CTLA-4 signaling pathway inhibitor is a small molecule that interacts with CTLA-4. In another embodiment, the CTLA-4 inhibitor is an anti-CTLA-4 antibody. In another embodiment, the immune checkpoint inhibitor is a CD40 signaling pathway inhibitor. In another embodiment, the immune checkpoint inhibitor is any other antigen-presenting cell:Tcell signaling pathway inhibitor known in the art.

It will be appreciated by the skilled artisan that any immune checkpoint protein known in the art can be targeted by an immune check point inhibitor. An immune checkpoint protein may be selected from, but is not limited to the following: programmed cell death protein 1 (PD1), T cell membrane protein 3 (TIM3), adenosine A2a receptor (A2aR) and lymphocyte activation gene 3 (LAG3), killer immunoglobulin receptor (KIR) or cytotoxic T-lymphocyte antigen-4 (CTLA-4). In another embodiment, the checkpoint inhibitor protein is one belonging to the B7/CD28 receptor superfamily.

In one embodiment, the T cell stimulator is an an antigen presenting cell (APC)/T cell agonist. In another embodiment, the T cell stimulator is a CD134 or a ligand thereof or a fragment thereof, a CD-137 or a ligand thereof or a fragment thereof, or an Includible T cell costimulator (ICOS) or a ligand thereof or a fragment thereof.

In one embodiment, the methods provided herein further comprise the step of co-administering an immunogenic composition provided herein with a cytokine that enhances an anti-tumor immune response in said subject. Cytokines that serve to enhance an immune response are well known and will be appreciated by the skilled artisan to include, type I interferons (IFN-α/IFN-β), TNF-α, IL-1, IL-4, IL-12, INF-γ, and any other cytokine known to enhance an immune response. In another embodiment, the cytokine is an inflammatory cytokine. In another embodiment, an immunogenic composition comprises cytokine known in the art or as provided herein. In another embodiment, administration of a cytokine may be prior to administration of an immunogenic composition provided herein. In another embodiment, administration of a cytokine may be concurrent with administration of an immunogenic composition provided herein. In another embodiment, administration of a cytokine may be after administration of an immunogenic composition as provided herein.

In one embodiment, the methods provided herein further comprise the step of co-administering an immunogenic composition provided herein with a indoleamine 2,3-dioxygenase (IDO) pathway inhibitor. IDO pathway inhibitors comprise small molecules that bind or interact with IDO, or an anti-IDO antibody. IDO pathway inhibitors for use in the present invention include any IDO pathway inhibitor known in the art, including but not limited to, 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody or a small molecule IDO inhibitor. In another embodiment, administration of an IDO pathway inhibitor may be prior to administration of an immunogenic composition provided herein. In another embodiment, administration of an IDO pathway inhibitor may be concurrent with administration of an immunogenic composition provided herein. In another embodiment, administration of any IDO pathway inhibitor may be after administration of an immunogenic composition as provided herein.

In another embodiment, the compositions and methods provided herein are also used in conjunction with, prior to, or following a chemotherapeutic or radiotherapeutic regiment. In another embodiment, IDO inhibition enhances the efficiency of chemotherapeutic agents.

In one embodiment, the methods provided herein further comprise the step of co-administering an immunogenic composition provided herein with a tumor kinase inhibitor that enhances an anti-tumor immune response in said subject. Tumor kinase inhibitors (TKIs) serve to interfere with specific cell signaling pathways and thus allow target-specific therapy for selected malignancies. TKI's are well known and will be appreciated by the skilled artisan to include those set forth in Table 1 below and any other TKI known to enhance an anti-tumor immune response. In another embodiment, administration of a TKI may be prior to administration of an immunogenic composition provided herein. In another embodiment, administration of a TKI may be concurrent with administration of an immunogenic composition provided herein. In another embodiment, administration of a TKI may be after administration of an immunogenic composition as provided herein.

TABLE 1

| Name | Target | Class |
|---|---|---|
| Afatinib | EGFR/ErbB2 | Small molecule |
| Axitinib | VEGFR1/VEGFR2/VEGFR3/ | Small molecule |
| | PDGFRB/c-KIT | |
| Bevacizumab | VEGF | Monoclonal antibody |
| Bosutinib | BcrAbl/SRC | Small molecule |
| Cetuximab | ErbB1 | Monoclonal antibody |
| Crizotinib | ALK/Met | Small molecule |
| Dasatinib | multiple targets | Small molecule |
| Erlotinib | ErbB1 | Small molecule |
| Fostamatinib | Syk | Small molecule |
| Gefitinib | EGFR | Small molecule |
| Ibrutinib | BTK | Small molecule |
| Imatinib | Bcr-Abl | Small molecule |
| Lapatinib | ErbB1/ErbB2 | Small molecule |
| Lenvatinib | VEGFR2/VEGFR2 | Small molecule |
| Mubritinib | N/A | Small molecule |
| Nilotinib | Bcr-Abl | Small molecule |
| Panitumumab | EGFR | Monoclonal antibody |
| Pazopanib | VEGFR2/PDGFR/c-kit | Small molecule |
| Pegaptanib | VEGF | RNA Aptamer |
| Ranibizumab | VEGF | Monoclonal antibody |
| Ruxolitinib | JAK | Small molecule |
| Sorafenib | multiple targets | Small molecule |
| SU6656 | multiple targets | Small molecule |
| Sunitinib | multiple targets | Small molecule |
| Tofacitinib | JAK | Small molecule |
| Trastuzumab | Erb2 | Monoclonal antibody |
| Vandetanib | RET/VEGFR/EGFR | Small molecule |
| Vemurafenib | BRAF | Small molecule |

In one embodiment, any of the above compounds or provided herein may be used in the present invention in combination with a chemotherapy, radiation or surgery regiment.

It will be well appreciated an “immunogenic composition” may comprise the recombinant Listeria provided herein, and an adjuvant, an immune checkpoint protein inhibitor, a T-cell stimulator, a TKI, or a cytokine, or any combination thereof. In another embodiment, an immunogenic composition comprises a recombinant Listeria provided herein. In another embodiment, an immunogenic composition comprises an adjuvant known in the art or as provided herein. In another embodiment, an immunogenic composition comprises an immune checkpoint inhibitor known in the art or as provided herein. In another embodiment, an immunogenic composition comprises an immune checkpoint inhibitor and a T-cell stimulator known in the art or as provided herein. In another embodiment, an immunogenic composition comprises a T-cell stimulator known in the art or as provided herein. It is also to be understood that such compositions enhance an immune response, or increase a T effector cell to regulatory T cell ratio or elicit an anti-tumor immune response, as further provided herein.

Following the administration of the immunogenic compositions provided herein, the methods provided herein induce the expansion of T effector cells in peripheral lymphoid organs leading to an enhanced presence of T effector cells at the tumor site. In another embodiment, the methods provided herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs, as demonstrated herein (see Examples). It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites. In another embodiment, methods of this invention eliciting an enhanced anti-tumor T cell response comprise an immune response comprising a decrease in the frequency of T regulatory cells (Tregs) in the spleen and the tumor microenvironment. In another embodiment, methods of this invention eliciting an enhanced anti-tumor T cell response comprise an immune response comprising a decrease in the frequency of myeloid derived suppressor cells (MDSCs) in the spleen and tumor microenvironment.

In one embodiment, combining the attenuated recombinant Listeria strains that express a fusion protein of truncated LLO and a heterologous antigen with a recombinant Listeria expressing the same antigen leads to complete tumor regression.

In another embodiment, a recombinant nucleic acid of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto. It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein”, “heterologous protein antigen”, “protein antigen”, “antigen”, and the like.

It will be appreciated by the skilled artisan that the term “episomal expression vector” compasses a nucleic acid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P. B. G. M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as provided herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as provided herein.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). Each method represents a separate embodiment of the methods and compositions as provided herein.

In one embodiment, the term “attenuation,” as used herein, is meant a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intraperitonealy, intra-ventricularly, intra-cranially, intra-vaginally or intra-tumorally.

In another embodiment of the methods and compositions provided herein, the vaccines or compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the vaccines or compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In one embodiment, the vaccines of the methods and compositions as provided herein may be administered to a host vertebrate animal, preferably a mammal, and more preferably a human, either alone or in combination with a pharmaceutically acceptable carrier. In another embodiment, the vaccine is administered in an amount effective to induce an immune response to the Listeria strain itself or to a heterologous antigen which the Listeria species has been modified to express. In another embodiment, the amount of vaccine or immunogenic composition to be administered may be routinely determined by one of skill in the art when in possession of the present disclosure. In another embodiment, a pharmaceutically acceptable carrier may include, but is not limited to, sterile distilled water, saline, phosphate buffered solutions or bicarbonate buffered solutions. In another embodiment, the pharmaceutically acceptable carrier selected and the amount of carrier to be used will depend upon several factors including the mode of administration, the strain of Listeria and the age and disease state of the vaccine. In another embodiment, administration of the vaccine may be by an oral route, or it may be parenteral, intranasal, intramuscular, intravascular, intrarectal, intraperitoneal, or any one of a variety of well-known routes of administration. In another embodiment, the route of administration may be selected in accordance with the type of infectious agent or tumor to be treated.

In another embodiment, the present invention provides a method of treating, suppressing, or inhibiting at least one tumor in a subject comprising administering the immunogenic composition provided herein.

In another embodiment, the present invention provides a kit for conveniently practicing the methods as provided herein comprising one or more Listeria strains as provided herein, an applicator, and instructional material that describes how to use the kit components in practicing the methods as provided herein.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequalae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

Materials and Methods (Examples 1 to 7):

Mice

C57BL/6 mice, female, 6-8-week-old (unless stated), were purchased from Frederick National Laboratory for Cancer Research (FNLCR). Mice were housed in the Animal Facility of National Cancer Institute, Bethesda. Protocols for use of experimental mice were approved by the Animal Care and Use Committee at National Institutes of Health.

Cell Line

TC-1 cells, which express low levels of E6 and E7, was derived from primary C57BL/6 mice lung epithelial cells by transformation with HPV-16 E6 and E7 and activated ras oncogene. The cells were grown in RPMI 1640, supplemented with 10% FBS, 100 U/ml penicillin, 100 μg/ml streptomycin, 2 mM L-glutamine, 1 mM sodium pyruvate, 100 μM nonessential amino acids, and 0.4 mg/ml G418 at 37° C. with 5% CO₂.

L. monocytogenes Strains

LmddA-LLO-E7 and its controls LmddA-LLO and LmddA were generated in Advaxis Inc (Princeton, N.J.). The dal dat ΔactA strain (LmddA) was constructed from the dal dat strain, which is based on Lm wild-type strain 10403S with a streptomycin resistance gene integrated into the chromosome. With dal, dat, and actA mutated, LmddA is highly attenuated. LmddA-LLO-E7 strain was constructed by transformation of LmddA with pTV3 plasmid after deletion of prfA, as well as the chloramphenicol resistance gene in the plasmid. Expression and secretion of LLO-E7 fusion protein was confirmed in the culture supernatants of LmddA-LLO-E7 strain by Western blotting as previously described. Construction of LmddA-LLO control strain was similar as that of LmddA-LLO-E7 strain but both prfA and E7 were deleted in pTV3 plasmid. Lm wild-type strain 10403S and some mutant strains, including Δhly, Δhly::pfo, and hly::Tn917-lac (pAM401-hly) were kindly provided by Dr. D. Portnoy (University of California, Berkeley, Calif.). The strain hly::Tn917-lac is a nonhemolytic mutant of wild-type Lm, in which the Tn917-lac fusion gene is inserted into the hly gene (the gene encoding LLO) to disrupt LLO hemolytic activity. When this mutant is transfected with a plasmid that expresses LLO (pAM401-hly), it gains hemolytic activity again since it has LLO. Lm-E7 strain, in which the full length of E7 gene was integrated into Lm chromosome, was kindly provided by Dr. Y. Paterson (University of Pennsylvania, Philadelphia, Pa.). Bacteria were cultured in brain heart infusion medium plus streptomycin (100 μg/ml) and in presence or absence of D-alanine (100 μg/ml).

Reagents

Fluorescence conjugated anti-mouse antibodies CD4-PerCP-Cy5.5 (GK1.5) and CD8-Brillient Violet 421 (53-6.7) were from Biolegend (San Diego, Calif.). FoxP3-FITC (FJK-16s) was from eBioscience (San Diego, Calif.). H-2D^{b} tetramers loaded with the E7 peptide (RAHYNIVTF) SEQ ID NO: 11 was kindly provided by the National Institute of Allergy and Infectious Diseases Tetramer Core Facility and the National Institutes of Health AIDS Research and Reference Reagent Program. CountBright™ absolute counting beads were from Life Technologies (Grand Island, N.Y.).

Tumor Inoculation and Mice Vaccination

TC-1 cells (10⁵ cells/mouse) were implanted s.c. in the right flank of mice on day 0. On day 10, when tumor became 5-6 mm in diameter, mice were injected i.p. with LmddA-LLO-E7 vaccine or proper controls at a dose of 0.1 LD50. Vaccination was boosted on day 17. Tumor was measured twice a week using an electronic caliper and tumor size was calculated by the formula: length×width×width/2. Mice were euthanized when tumor reached 2.0 cm in diameter.

Flow Cytometry

Mouse splenocytes or cells harvested from tumor were stained with CD4-PerCP-Cy5.5, CD8-Brillient Violet 421, and H-2D^{b} E7 tetramer-APC for 30 min. Cells were fixed, permeabilized, and stained with FoxP3-FITC overnight. Cells were analyzed by flow cytometry. A lymphocyte gate was set where Tregs were identified as CD4+FoxP3+. CountBright™ absolute counting beads were added for counting absolute cell numbers.

Adoptive Transfer of CD4+CD25+ Tregs

CD4+CD25+ T cells were isolated from mouse spleens by Dynal® CD4+CD25+Treg Kit (Life Technologies, Grand Island, N.Y.). Cells were injected i.v. into TC-1 tumor-bearing mice at day 9 post tumor cell inoculation. One day after Treg transfer, mice were immunized i.p. with LmddA-LLO-E7 (0.1 LD50) twice at one week interval. Tumor growth was monitored.

Statistics

The data were analyzed using the nonparametric Mann-Whitney test. Significance was determined at P<0.05.

### Example 1: LmddA-LLO-E7 Induces Regression of Established TC-1 Tumors Accompanied by Treg Frequency Decrease

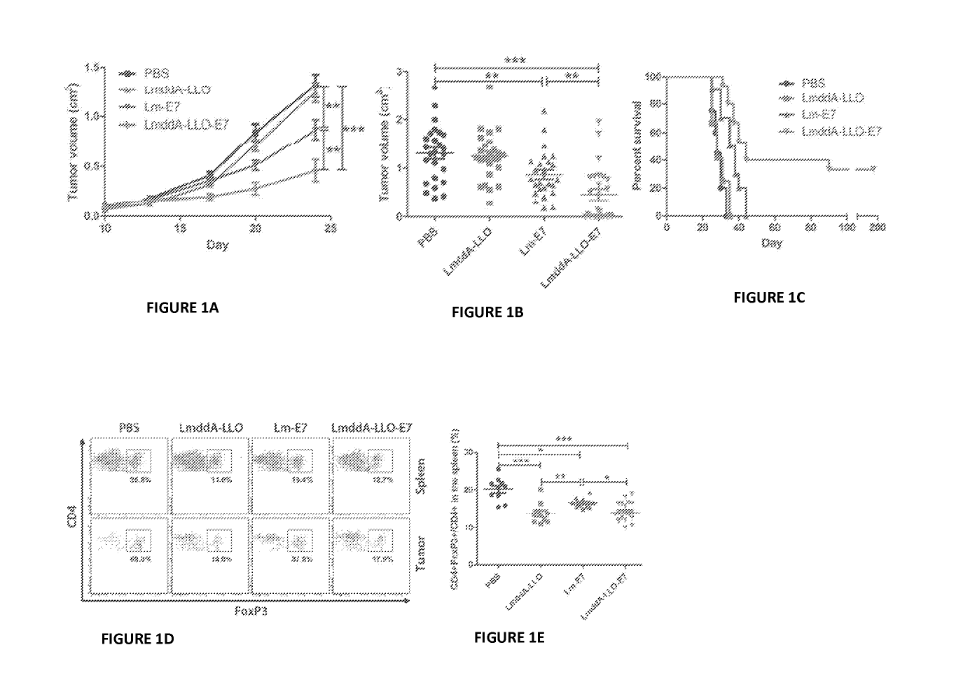

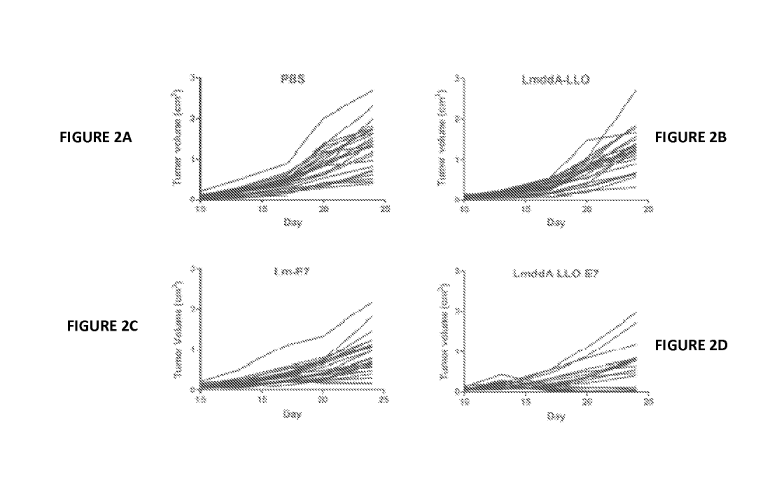

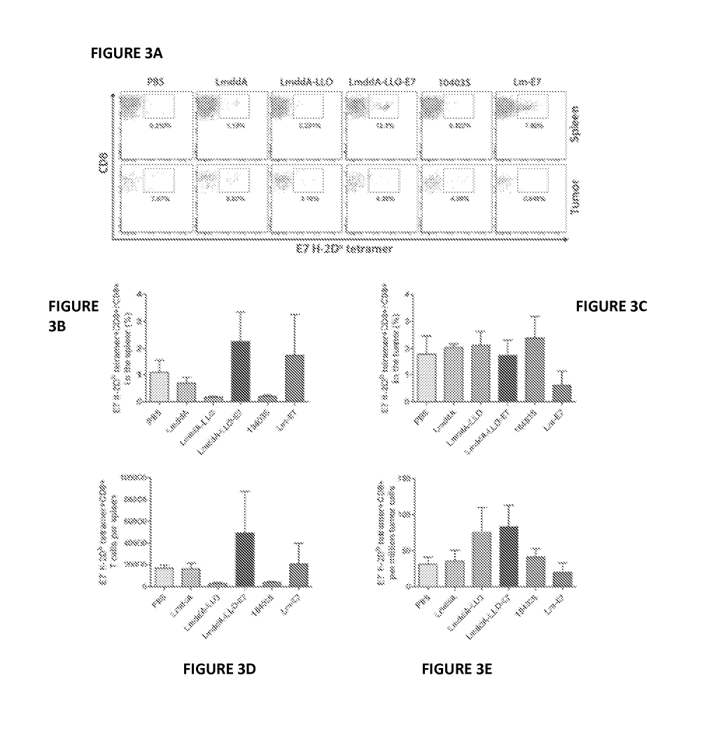

It was previously reported that a Lm-based vaccine, Lm-LLO-E7, where a fusion protein LLO-E7, as well as PrfA, is expressed episomally in a prfA negative strain of Listeria XFL-7, induced complete regression of established TC-1 tumors. Here the antitumor activity of another highly attenuated Lm-based vaccine, LmddA-LLO-E7, which produces the fusion protein LLO-E7 by a plasmid in a dal, dat, and actA mutated Lm strain, was investigated. LmddA-LLO-E7 is more attenuated compared to Lm-LLO-E7, since the chloramphenicol resistance gene and PrfA have been removed from the plasmid. It was observed that similar to Lm-LLO-E7, LmddA-LLO-E7 significantly inhibited the growth of established TC-1 tumors (FIG. 1A and FIG. 1B, FIG. 2). Tumor completely regressed in approximately 40% of TC-1 tumor-bearing mice after vaccination with LmddA-LLO-E7 twice (FIG. 1B and FIG. 2). Except for one mouse that relapsed and died at 3 months, the others that showed tumor regression (33% of total animals) survived at least 6 months without relapse (FIG. 1C). Although Lm-E7 slowed down TC-1 tumor growth, it failed to induce complete tumor regression (FIG. 1A and FIG. 1B and FIG. 2). LmddA-LLO (without E7) was unable to significantly inhibit TC-1 tumor growth (FIG. 1A and FIG. 1B and FIG. 2), suggesting that innate immune response is not sufficient to eradicate TC-1 tumor cells. LmddA-LLO-E7 and Lm-E7 induced similar H-2D^{b} E7 tetramer+CD8+ T cell response in the spleen (FIG. 3A-upper panel, FIG. 3B, and FIG. 3D), which was consistent with previous finding. CD4+FoxP3+ Tregs were then analyzed. Unexpectedly, it was observed that LmddA-LLO-E7, Lm-E7, and LmddA-LLO, all significantly decreased Treg frequency in the spleen and more dramatically in the tumor compared to PBS control, though LmddA-LLO-E7 and LmddA-LLO decreased the frequency more than did Lm-E7 (FIGS. 1D-1H).

### Example 2: Lm is Sufficient to Induce Decrease of Treg Frequency

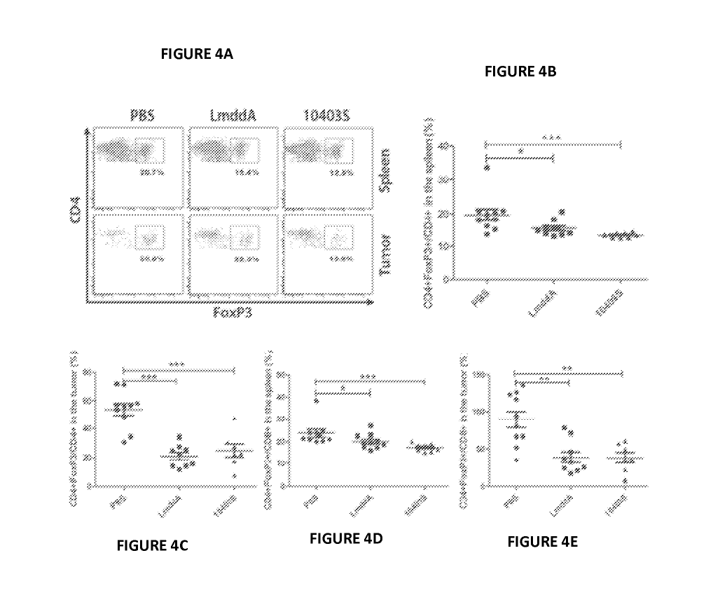

Initially, it was suspected that the decrease of Treg frequency was mediated by the truncated LLO. But Lm-E7, without expression of the truncated LLO, was also able to decrease Treg frequency (FIGS. 1D-1H). This observation suggests that Lm might be able to decrease Treg frequency. Indeed, both LmddA, the vector control for LmddA-LLO-E7, and 10403S, a wild-type Lm strain and the vector control for Lm-E7, significantly decreased Treg frequency in the spleen and more so in the tumor (FIG. 4).

### Example 3: Lm Decreases Treg Frequency by Preferentially Inducing CD4+FoxP3− T Cell and CD8+ T Cell Expansion

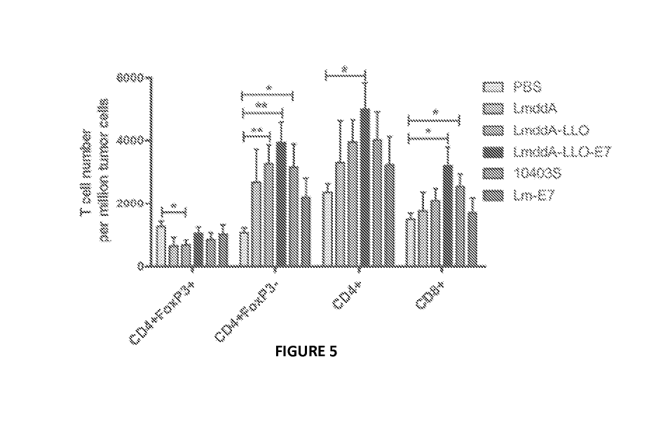

A relative Treg frequency (proportion of total T cells) is determined not only by the number of Tregs but also by the number of CD4+FoxP3− T cells and CD8+ T cells. To investigate how Lm decreases Treg frequency, CD4+FoxP3+Treg, CD4+FoxP3− T cell and CD8+ T cell number were quantified in TC-1 tumor-bearing mice treated with LmddA-LLO-E7, LmddA-LLO, LmddA, Lm-E7, or Lm (10403S). As shown in FIG. 5, surprisingly, it was found that LmddA did not markedly change the number of CD4+FoxP3+ T cells in the tumor. It actually increased CD4+FoxP3− T cells and CD8+ T cells, thus decreasing Treg frequency proportionately. Episomal expression of a truncated LLO in LmddA-LLO and LmddA-LLO-E7 further increased CD4+FoxP3− T cells and CD8+ T cells, thus decreasing CD4+FoxP3+ T cell frequency more. Wild-type Lm 10403S and Lm-E7 also induced an increase in CD4+FoxP3− T cells and CD8+ T cells while not significantly changing CD4+FoxP3+ T cell number. Lm-LLO-E7 significantly increased the density of CD4+FoxP3-T cells and CD8+ T cells in the tumor. These results demonstrate that Lm preferentially induces CD4+FoxP3− T cell and CD8+ T cell expansion to decrease CD4+FoxP3+ T cell frequency.

### Example 4: Lm-Induced Expansion of CD4+FoxP3− T Cells and CD8+ T Cells is Dependent on and Mediated by LLO

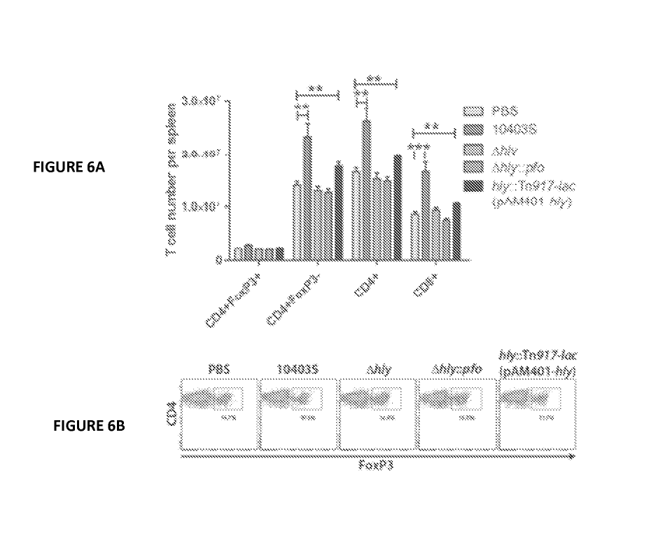

LLO, encoded by the hly gene, is a pore-forming cytolysin by which Lm can escape from a host cell phagosomal vacuole into the cytoplasm. Since LmddA-LLO-E7, Lm-E7 and all their controls produce LLO, a LLO-deficient Lm mutant derived from 10403S, in which hly gene is deleted using a shuttle vector followed by homologous recombination, was used to study if LLO plays a role in inducing expansion of CD4+FoxP3− T cells and CD8+ T cells. It was found that Δhly Lm was unable to increase CD4+FoxP3− T cells and CD8+ T cells in the spleen of mice on day 7 after a single administration (FIG. 6A), indicating that induction of CD4+FoxP3− T cell and CD8+ T cell expansion is dependent on LLO. This could be a direct effect of LLO or a requirement to escape the phagolysosome. To address this question, an LM with LLO replaced by PFO was studied. Perfringolysin O (PFO), produced by Clostridium perfringens, is 43% identical in amino acids with LLO and can also lyse the vacuolar membrane. The pfo gene, encoding PFO under the control of hly promoter, was recombined into the chromosome of the Δhly strain to form Δhly::pfo strain. Although Δhly::pfo was able to escape from phagocytosis into the cytoplasm, it was unable to increase CD4+FoxP3− T cells and CD8+ T cells in the mouse spleen (FIG. 6A). In contrast, hly::Tn917-lac (pAM401-hly), a nonhemolytic Tn917-lac mutant of wild-type Lm (in which Tn917-lac fusion gene is inserted into the hly gene to disrupt LLO hemolytic activity) transformed with a LLO expressing plasmid pAM401-hly, induced expansion of mouse splenic CD4+FoxP3− T cells and CD8+ T cells (FIG. 6A). These results suggest that expansion of CD4+FoxP3− T cells and CD8+ T cells is directly mediated by LLO. Since Lm did not induce CD4+FoxP3+ T cell expansion significantly, Lm-induced Treg decrease in frequency resulted from the increase of CD4+FoxP3− T cells and CD8+ T cells (FIGS. 6A-6D).

### Example 5: Episomal Expression of a Truncated LLO in LmddA Induces Expansion of CD4+FoxP3− T Cells and CD8+ T Cells to a Higher Level

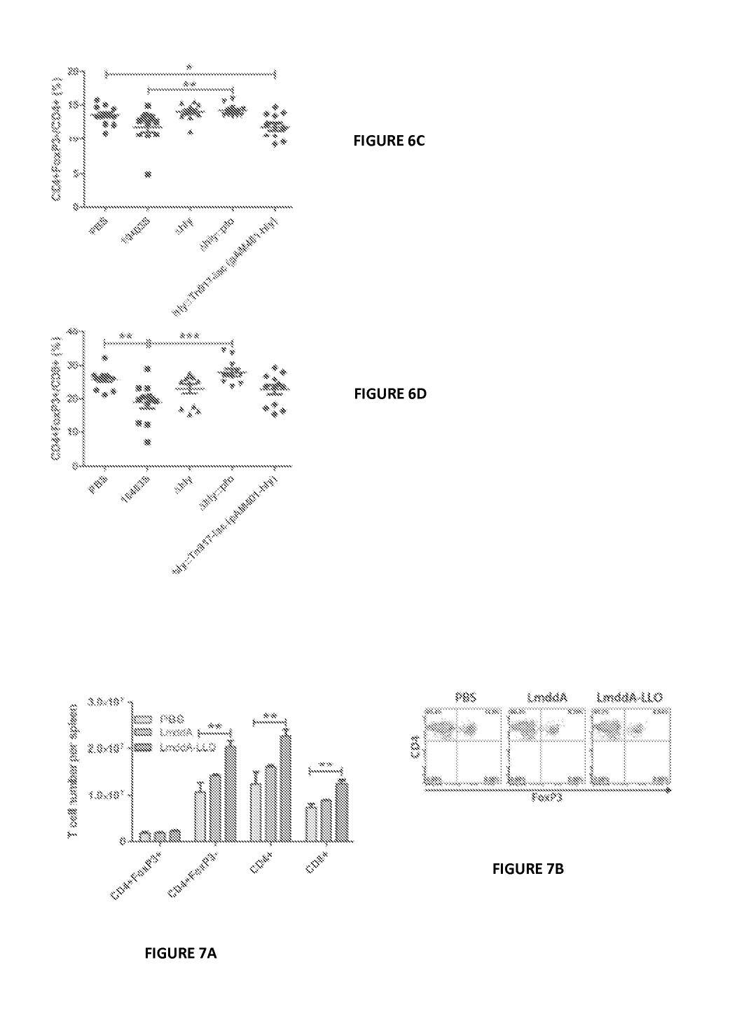

Next LmddA and LmddA-LLO were compared, in which the latter produces a truncated LLO episomally by a plasmid, in induction of T cell proliferation in healthy, non-tumor-bearing mice. It was found that LmddA was able to slightly increase CD4+FoxP3− T cell and CD8+ T cell number in the spleen of mice at day 7 after a single administration, but LmddA-LLO further induced such an increase to a higher level (FIG. 7A). In contrast, CD4+FoxP3+ T cell number was not significantly changed after LmddA or LmddA-LLO infection (FIG. 17A). These resulted in a significant decrease of Tregs in proportion after LmddA-LLO administration compared to PBS control (FIGS. 7B-7D). The presence of the cell proliferation marker Ki-67 in these cells, was examined. LmddA increased the frequency and absolute number of Ki-67+CD4+FoxP3− T cells and Ki-67+CD8+ T cells, but LmddA-LLO increased the number to a greater extent (FIGS. 7E-7G). The level of Ki-67 expression in CD4+FoxP3− T cell and CD8+ T cells was also increased accordingly (FIG. 7H). In contrast, the frequency and absolute number of Ki-67+CD4+FoxP3+ T cells and Ki-67 expression in CD4+FoxP3+ T cells was not markedly changed, indicating LmddA and LmddA-LLO did not induce their proliferation.

### Example 6: The Combination of Lm-E7 and LmddA-LLO Induces Regression of Established TC-1 Tumors

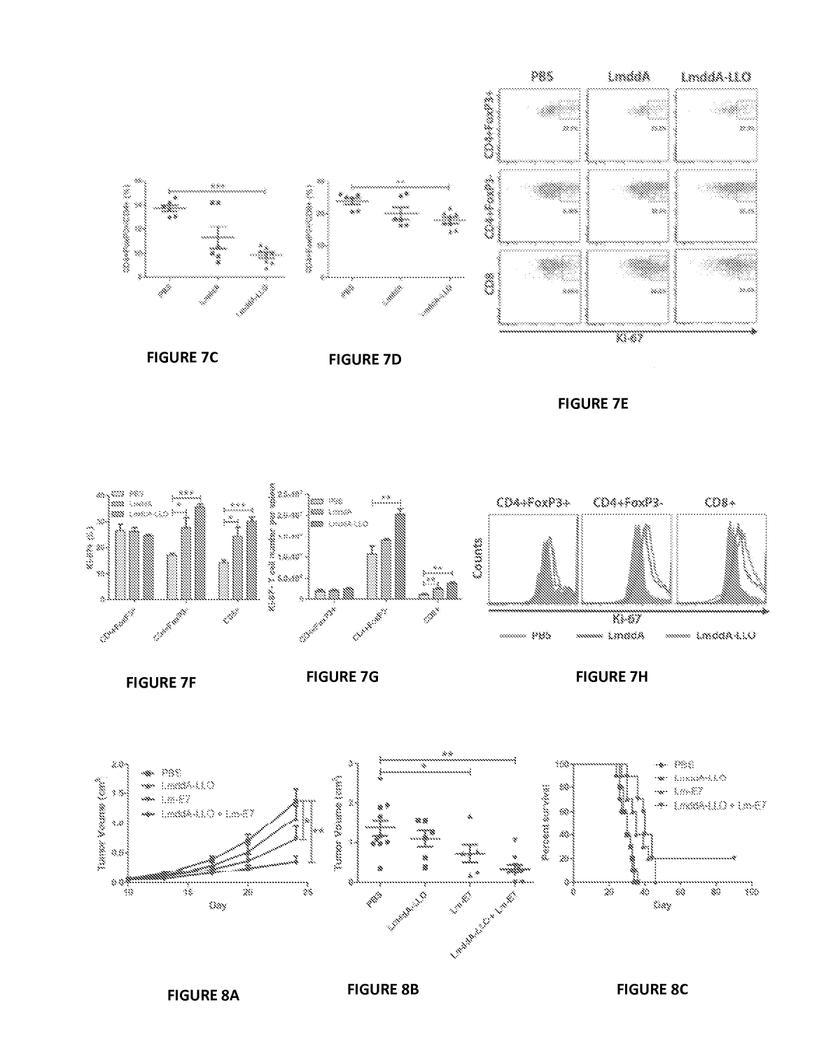

The Lm-E7 vaccine alone did not induce much expansion of CD4+FoxP3− T cells and CD8+ T cells (FIG. 5). This may account for its failure in induction of TC-1 tumor regression. Since LmddA-LLO induced CD4+FoxP3− T cell and CD8+ T cell expansion (FIG. 5 and FIG. 7A), it is conceivable that the anti-tumor effect of Lm-E7 may be improved in the presence of LmddA-LLO. Indeed, the combination of Lm-E7 and LmddA-LLO induced nearly complete regression of established TC-1 tumors (FIGS. 8A-8C). In contrast, addition of LmddA failed to augment Lm-E7-induced anti-tumor activity (Data not shown), indicating the importance of the truncated non-hemolytic LLO in improving the anti-tumor efficacy of Lm-E7 vaccine. As expected, CD4+FoxP3− T cell and CD8+ T cell number was significantly increased in the spleen of the combination group mice compared with those treated with Lm-E7 or PBS (FIG. 8D). Again, because CD4+FoxP3+ number was relatively unchanged, the increase of CD4+FoxP3− T cell and CD8+ T cell number to a higher level by combined Lm-E7 and LmddA-LLO resulted in a greater decrease in the CD4+FoxP3+ T cell proportion (FIGS. 8E-8G).

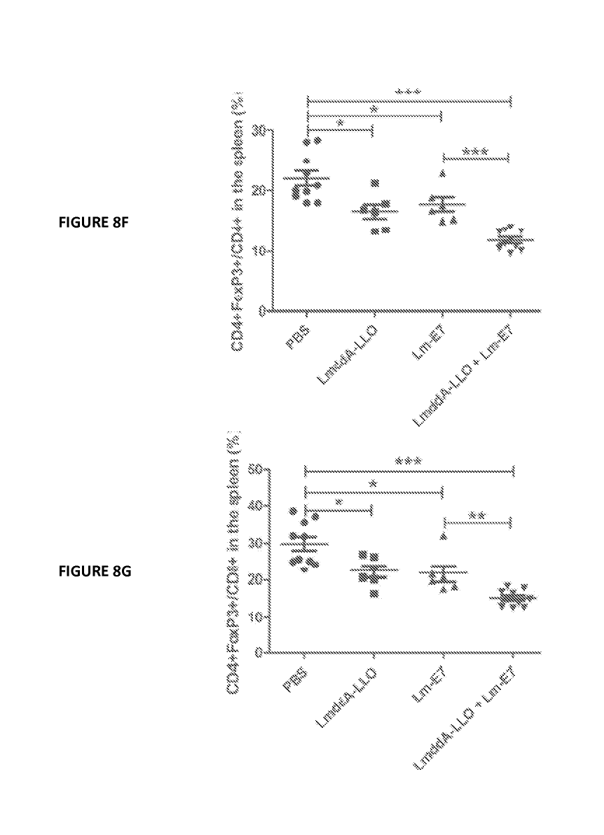

Moreover, LmddA was also co-administered with Lm-E7 as a control to determine the role the non-hemolytic truncated LLO played during co-administration of LmddA-LLO and Lm-E7. It was observed that the addition of the LmddA strain failed to augment the Lm-E7 induced anti-tumor activity, indicates that the endogenous LLO produced by LmddA could not assist Lm-E7-induced anti-tumor activity (FIG. 10).

### Example 7: Adoptive Transfer of Tregs Compromises the Anti-Tumor Efficacy of LmddA-LLO-E7 Against Established TC-1 Tumors

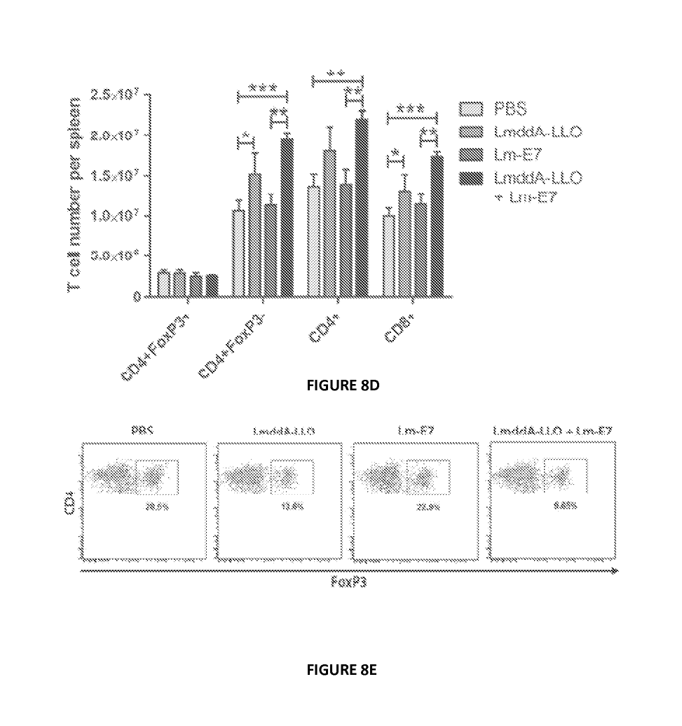

LmddA-LLO-E7 did not significantly change Treg numbers, although it decreased Treg frequency (FIGS. 1D-1H). The ratio of Tregs to CD4+FoxP3− T cells or to CD8+ T cells has been a well-accepted parameter to determine Treg suppressive ability. To determine whether the Treg proportion has any impact on the anti-tumor efficacy of LmddA-LLO-E7, CD4+CD25+ Tregs from naïve C57BL/6 mice were isolated and injected them i.v. into TC-1 tumor-bearing mice, which were followed by LmddA-LLO-E7 vaccination. LmddA-LLO-E7 significantly inhibited TC-1 tumor growth in the mice without adoptive transfer of Tregs (FIG. 9A and FIG. 9B). However, in the mice given Tregs, LmddA-LLO-E7 was unable to significantly inhibit TC-1 tumor growth (FIG. 9A and FIG. 9B). Mice receiving Tregs showed a slight increase of Treg number in the spleen but more decrease in the tumor. On the other hand, mice receiving Tregs had fewer CD4+FoxP3− T cells and CD8+ T cells after being vaccinated with LmddA-LLO-E7 compared to the LmddA-LLO-E7 control, indicating adoptive transfer of Tregs inhibits CD4+FoxP3− T cell and CD8+ T cell expansion (FIGS. 9, F and G). These together resulted in the increase of Treg frequency in the Treg-recipient mice (FIGS. 9C-9E).

It is well-known that tumor antigen-specific CTLs play dominant roles in killing tumor cells, and Lm, as an intracellular bacteria, can deliver antigens associated with MHC class I molecules to activate CTLs. However, why did two Lm-based vaccines, Lm-LLO-E7 and Lm-E7, induce similar levels of HPV E7-specific CTLs in the spleen but nevertheless exhibit distinct anti-tumor activity, with the former inducing a much stronger anti-tumor effect (FIGS. 1A-1C, FIG. 2, FIG. 3). It is no doubt that CD8+ T cells participate in killing tumor cells, as their depletion abrogated Lm-LLO-E7-induced tumor regression. It is also clear that a certain level of tumor-antigen specific CTLs is necessary for killing tumor cells, as LmddA-LLO, which lacks E7 expression, was unable to significantly inhibit TC-1 tumor growth (FIGS. 1A-1C and FIG. 2). It has been proposed that Lm-E7 induced an increase of Tregs to suppress the host immune response, thus compromising its anti-tumor immunity. However, it was found that actually both Lm-E7 and LmddA-LLO-E7 decreased Treg frequency in a TC-1 tumor model compared to PBS control (FIGS. 1D-1H). What is more, it was found that neither Lm-E7 nor LmddA-LLO-E7 significantly increased Treg total number in TC-1 tumor after vaccination (FIG. 5).

In fact, it was found that a major difference between LmddA-LLO-E7 and Lm-E7 is that the former was able to induce a marked increase of CD4+FoxP3− T cell and CD8+ T cell number while the latter induced a increase to a much less degree (FIG. 5). This explains why LmddA-LLO-E7 decreased Treg percentage to a greater degree than Lm-E7 (FIGS. 1D-1H). It was observed that Lm vector was sufficient to increase CD4+FoxP3− T cell and CD8+ T cell number. However, with episomal expression of a truncated LLO, Lm increased CD4+FoxP3− T cell and CD8+ T cell number dramatically to a higher level, thus decreasing Treg frequency even further (FIG. 7). Thus, it is conceivable that LLO plays a critical role in inducing increase of CD4+FoxP3− T cell and CD8+ T cell number. Indeed, LLO is not only necessary for L. monocytogenes to escape from the phagosome but also directly causes CD4+FoxP3− T cell and CD8+ T cell expansion, as neither a LLO-minus (Δhly) L. monocytogenes strain nor a Δhly::pfo strain, which expresses PFO that enables Lm to enter the cytoplasm, succeeded in inducing CD4+FoxP3− T cell and CD8+ T cell proliferation, but transformation of a nonhemolytic LLO mutant Lm strain with an LLO-expressing plasmid restored CD4+FoxP3− T cell and CD8+ T cell expansion (FIG. 6). LLO-induced CD4+FoxP3− T cell and CD8+ T cell expansion is unrelated to its hemolytic activity, as episomal expression of a nonhemolytic truncated LLO in LmddA greatly augmented CD4+FoxP3− T cell and CD8+ T cell expansion (FIG. 7). Although the expansion of both CD4+ T cell and CD8+ T cell responses by LLO appears to be an antigen-non-specific adjuvant effect, LLO may also contain immuno-dominant epitopes of these two cell types. Indeed, early studies identified that LLO bears two CD4+ T cell epitopes (residues 189-201 and residues 215-226, respectively) and one CD8+ T cell epitope (residues 91-99).

LmddA-LLO-E7's excellent anti-tumor effect is likely due to the fact that it induces a significant increase of CD4+FoxP3− T cells and CD8+ T cells. In contrast, the inability of Lm-E7 to induce marked increase of CD4+FoxP3− T cell and CD8+ T cell number accounts for its inefficiency in eradication of tumors, as the combination of Lm-E7 and LmddA-LLO, which dramatically increased CD4+FoxP3− T cell and CD8+ T cell number compared to Lm-E7 alone, induced nearly complete regression of established TC-1 tumors (FIG. 8). Our data indicate that the LmddA-LLO-E7-induced decrease in Treg frequency is the consequence of an increase in CD4+FoxP3− T cell and CD8+ T cell number. The ratio of Tregs to CD4+FoxP3− T cells or to CD8+ T cells is critical, to suppress the function of CD4+FoxP3− T cells and CD8+ T cells. Indeed, increasing the Treg ratio in vivo by adoptive transfer of Tregs to tumor-bearing mice followed by LmddA-LLO-E7 vaccination inhibited the expansion of CD4+FoxP3− T cells and CD8+ T cells and consequently compromised the vaccine's anti-tumor efficacy (FIG. 9).

Besides preferentially inducing the expansion of CD4+FoxP3− T cells and CD8+ T cells, the truncated non-hemolytic LLO makes other contributions to improving the anti-tumor efficacy of LmddA-LLO-E7 vaccine. It was observed that although Lm-E7 and LmddA-LLO-E7 induced similar expansion of E7-specific CD8+ T cells, but this is not the case in the tumor. With episomal expression of the truncated LLO (LmddA-LLO-E7), more E7-specific CD8+ T cells tended to be induced in the tumor (FIG. 3E). It was found that LmddA-LLO-E7 upregulated the expression of chemokine receptors CCR5 and CXCR3 on CD4+FoxP3− T cells and CD8+ T cells, but not on CD4+FoxP3+ T cells showing that CCR5 and CXCR3 are crucial for Th1 and CD8+ T cell trafficking. These results suggest that LLO induces CD4+FoxP3− T cell and CD8+T antigen-specific cell migration to the tumor microenvironment through upregulation of CCR5 and CXCR3. In addition, it is known that truncated LLO is required for the efficient secretion of the antigen from Lm, and antigens that are not secreted from the Lm vector result in the induction of less effective anti-tumor immunity. Hence, the lack of potent anti-tumor activity of the Lm-E7 vector might not only be due to the lack of effectively expanding the CD4+FoxP3− T cells and CD8+ T cells but also be due to the inefficient secretion of the antigen from Lm in context of an infected antigen presenting cell and the priming of an ineffective antigen-specific T cell response.

Overall, it was demonstrated that episomal expression of a nonhemolytic truncated LLO in a LmddA-LLO-E7 vaccine preferentially induces CD4+FoxP3− T cell and CD8+ T cell expansion, which enhances the vaccine's anti-tumor activity. In conclusion, the results show that many factors, like a certain level of antigen-specific CTLs, and of non-tumor antigen-specific CD4+FoxP3− T cells and CD8+ T cells, and a decreased Treg proportion, are all needed to trigger an effective anti-tumor immune response, and that this can be accomplished with the Listeria constructs provided herein. Further, this work indicates that LLO is a promising vaccine adjuvant in that it preferentially induces CD4+FoxP3− T cell and CD8+ T cell expansion, thus overall decreasing Treg frequency and favoring immune responses to kill tumor cells.

### Materials and Methods (Examples 8 to 12)

Animals, Cells Lines, Vaccine and Other Reagents

Six to eight weeks old female C57BL6 mice were purchased from NCI Frederick and kept under pathogen-free conditions. Mice were cared for under protocols approved by the NCI Animal Care and Use Committee. TC-1 cells that were derived by co-transfection of human papillomavirus strain 16 (HPV16) early proteins 6 and 7 (E6 and E7) and activated ras oncogene to primary C57BL/6 mouse lung epithelial cells were obtained from ATCC (Manassas, Va.), and cells were grown in RPMI 1640 supplemented with 10% FBS, penicillin and streptomycin (100 U/ml each) and L-glutamine (2 mM) at 37° C. with 5% CO₂. Listeria vaccine vectors with or without human papilloma virus-16 (HPV-16) E7 (Lm-LLO and Lm-LLO-E7) were provided by Advaxis Inc. Both Lm-LLO and Lm-LLO-E7 were injected intraperitonealy (i.p.) at 5×10⁶ CFU/mouse dose. The antiPD-1 monoclonal antibody was obtained from CureTech (Israel) and was injected intravenously (i.v.) at a dose of 50 g/mouse. All fluorescently labeled antibodies and appropriate isotype controls used for flow cytometry were purchased from BD Biosciences (San Jose, Calif.) or eBiosciences (San Diego, Calif.).

### Mouse and Human Dendritic Cell Isolation, Purification and Analysis of PD-L1 Expression

Mouse dendritic cells (DC) were isolated and purified from bone marrow as described earlier. To obtain human DC, monocytes were isolated from healthy adult blood donors (National Institute of Health, Blood bank). Briefly, peripheral blood mononuclear cells (PBMC) were isolated from gradient centrifugation using Ficoll-Paque Plus (Amersham Biosciences) and, after washing, allowed to adhere to tissue culture plates for 2 h at 37° C. Nonadherent cells were removed by washing, and the adherent monocytes were cultured in a plate at 37° C., 5% CO₂ in complete RPMI 1640 consisting of RPMI 1640, 2 mM L-glutamine, penicillin (100 U/ml), streptomycin (100 ug/ml), 10 mM HEPES, 10% fetal bovine serum, 10 mM nonessential amino acids, 1 mM sodium pyruvate, and 5×10⁻⁵ M2-mercaptoethanol. Cells were cultured in the presence of GM-CSF (1000 U/ml) and IL-4 (500 U/ml) for 4 days to become immature DCs. GM-CSF and IL-4 were added again along with fresh medium on day 3. The DC viability in cultures was assessed using the trypan blue exclusion protocol. Trypan blue-negative cells were considered alive. After culturing DCs from monocytes for 4-5 days, DCs were collected and transferred to 6 well plate (1×10⁶ cells/ml). Different concentrations of Lm-LLO or Lm-LLO-E7 were added to DCs culture (0, 10⁷, 10⁸, and 10⁹ CFU/ml) for an hour followed by adding gentamicin (50 ug/ml) to kill listeria, and cultured for 48 hr.

Both mouse and human DCs were stained with appropriate fluorescently labeled anti-PD-L1 antibody (PE anti-mouse PD-L1 and HFTC anti-human PD-L1). Isotype-matched mAbs were used as negative controls. The stained cells were analyzed using FACS Calibur cytometer and CellQuest software (BD Biosciences).

### Tumor Implantation and Treatment

The therapeutic experiments aimed to analyze tumor growth and survival were performed as described earlier. Briefly, mice were implanted with 50,000 TC-1 cells/mouse subcutaneous (s.c.) in the right flank on day 0. On day8 (tumorsize-3-4 mm in diameter), animals from appropriate groups (5 mice per group) were injected i.p. with Lm-LLO or Lm-LLO-E7 with or without anti-PD-1 Ab i.v. Mice were treated with vaccine and anti-PD-1 Ab one more time on day 15 after tumor implantation. Another group of mice remained non-treated. Tumors were measured every 3-4 days using digital calipers, and tumor volume was calculated using the formula V=(W2×L)/2, whereby V is volume, L is length (longer diameter) and W is width (shorter diameter). In these experiments mice were sacrificed when mice became moribund, tumors were ulcerated or tumor volume reached 1.5 cm 3. In immunologic experiments same groups of mice were treated similarly, except mice were sacrificed six days after the second treatment, on day 21. Spleens and tumors were isolated and analyzed for antigen-specific immune responses, CD8 T cells, Tregs and myeloid derived suppressor cells (MDSC).

Analysis of antigen-specific cellular immune responses, Tregs, MDSC in periphery and tumors ELISPOT was used to detect IFNγ production in E7-restimulated (10 μg/ml) splenocyte cultures from treated and control mice, as suggested by the manufacturer (BD Biosciences, San Jose, Calif.). A CTL Immunospot Analyzer (Cellular Technology Ltd., Shaker Heights, Ohio) was used to analyze spots. The number of spots from irrelevant peptide (hgp 10025-33—KVPRNQDWL (SEQ ID NO: 12)-Celtek Bioscience, Nashville, Tenn.) re-stimulated splenocytes were subtracted from E7-restimulated cultures. Tumor samples were processed using GentleMACS Dissociator and the solid tumor homogenization protocol, as suggested by the manufacturer (Miltenyi Biotec, Auburn, Calif.). The number of tumor-infiltrating CD8+, CD4+Foxp3+(Treg) and CD11b+Gr-1+(MDSC) cells were analyzed within CD45+ hematopoietic cell population using flow cytometry assay as was described earlier. The level of Treg cells and MDSC was also evaluated in spleens of tumor-bearing treated and control mice using the same flow cytometry assay.

### Statistical Analysis

All statistical parameters (average values, SD for PD-L1 expression on DC, tumor volumes, ELISPOT and peripheral and tumor-infiltrating cell analysis) and statistical significance between groups (for peripheral and tumor infiltrating cell analysis) were calculated using GraphPad Prism Software (San Diego, Calif.). Statistical significance between groups was determined by one-way ANOVA with Tukey's multiple comparison post-test (P<0.05 was considered statistically significant).

### Results

### Example 8: Infection of Murine DC with Lm-LLO and Lm-LLO-E7 Upregulates Surface PD-L1 Expression

It was previously demonstrated that mouse splenocytes infection with Lm results in significant upregulation of PDL1 expression on the majority of cells, and that the level of

PD-L1 expression was highest among CD11c+DC.

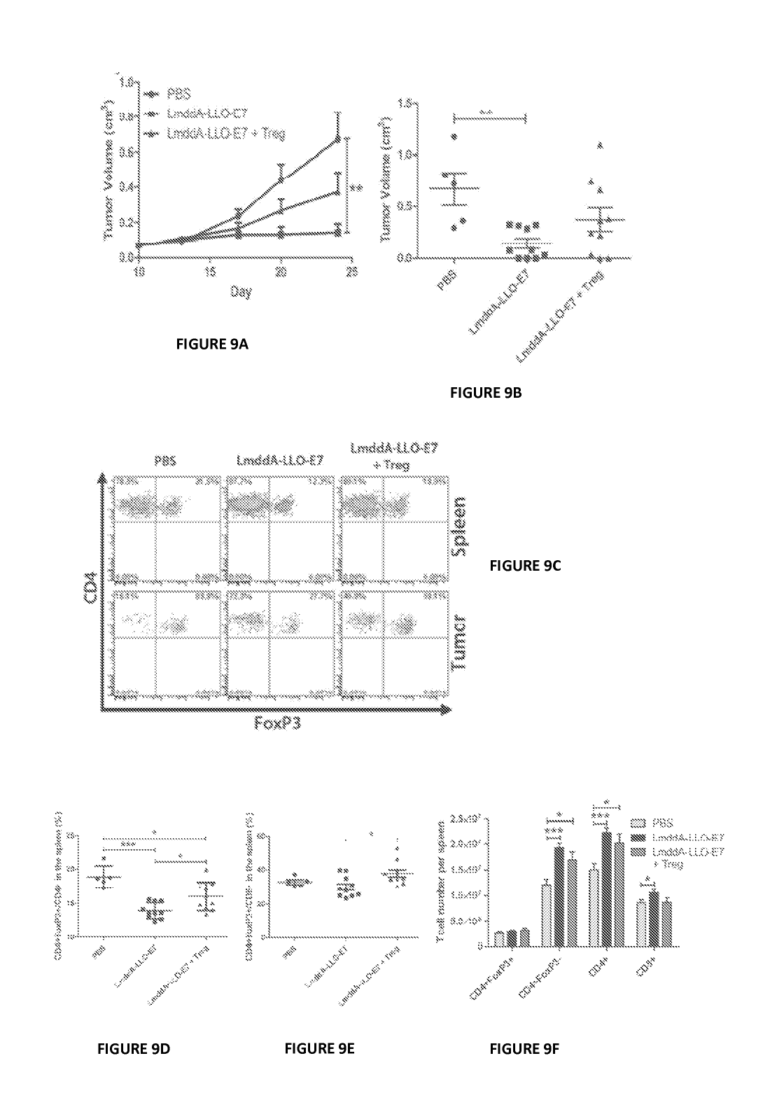

Considering the importance of DC in priming immune response and inhibitory role of PD-1/PD-L1 interaction, it was decided first to analyze the effect of Lm-LLO and LmLLO-E7 on the PD-L1 expression on DC. To avoid the influence of cell-cell interactions within the mixed cell population on the accuracy of results, the effect of different concentrations of Lm-LLO and Lm-LLO-E7 on PD-L1 expression on the surface of purified CD11c+ DCs was tested. As shown in FIG. 11, both Lm-LLO and LmLLO-E7 significantly upregulate PD-L1 expression at 10⁸ and 10⁹ CFU/ml doses in a dose dependent manner.

Importantly, there were no differences detected between Lm-LLO- and Lm-LLO-E7-induced PD-L1 upregulation on DC at any of the tested doses (FIG. 11), indicating that this effect is antigen-independent.

### Example 9: Anti-PD-1 Enhances Therapeutic Efficacy of Lm-LLO-E7 Vaccine

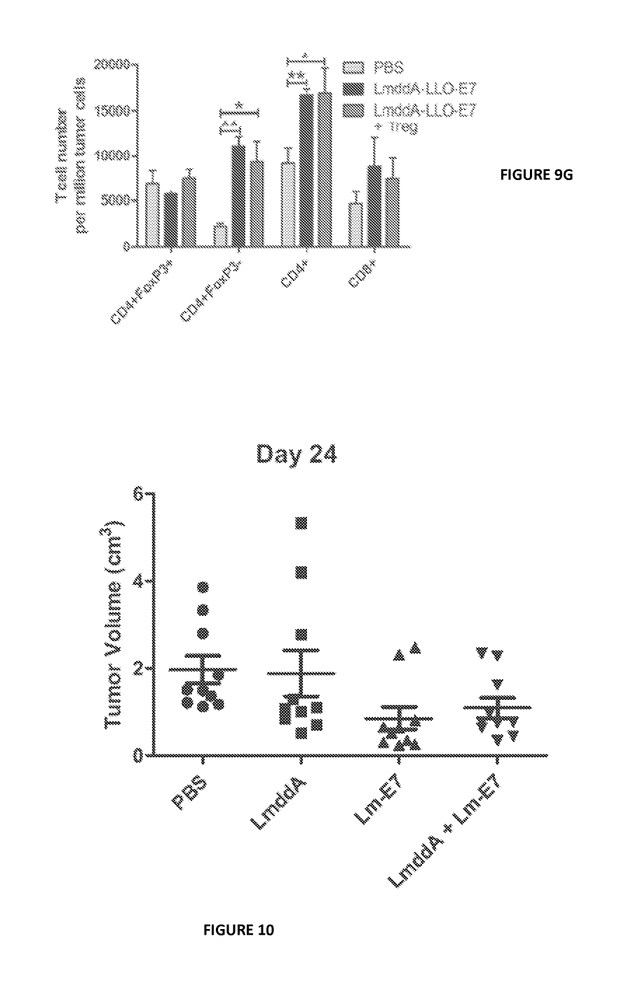

After confirming the effect of Lm-LLO and Lm-LLO-E7 on upregulation of PD-L1 expression on DC, and considering the inhibitory effect of PD-1/PD-L1 interaction it was hypothesize that combination of PD-1/PD-L1 blockade with Listeria-based vaccine could improve the anti-tumor efficacy of immunotherapy. To test this hypothesis the effect of anti-PD-1 Ab and Lm-LLO-E7 combination on tumor growth and survival of mice in TC-1 tumor model based on E7-expressing lung epithelial cells was evaluated. A low dose of Lm-LLO-E7, delayed treatment schedule and implanting a high number of tumor cells was deliberately used in order to minimize the effect of vaccine alone. Mice were implanted with 50,000 TC-1 cells s.c. on day 0, and on days 8 and 15 after tumor implantation mice were injected with Lm-LLO-E7 or Lm-LLO with or without anti-PD-1 Ab (FIG. 12A). Another group of mice remained non-treated.

While Lm-LLO-E7 vaccine alone resulted in slight inhibition of tumor growth, Lm-LLO-E7/anti-PD-1 combination significantly slowed tumor growth (FIG. 12B) and resulted in prolonged survival and complete tumor regression in 20% of treated mice (FIG. 12C). These experiments reveal that combination of antiPD-1 Ab with Lm-LLO-E7 vaccine is a feasible strategy resulting in tumor growth inhibition and improved survival even at stringent conditions that were used in these experiments.

### Example 10: Combination of Anti-PD-1 Ab and Lm-LLO-E7 Significantly Enhances Antigen-Specific Immune Responses and CD8 T Cell Infiltration into the Tumor

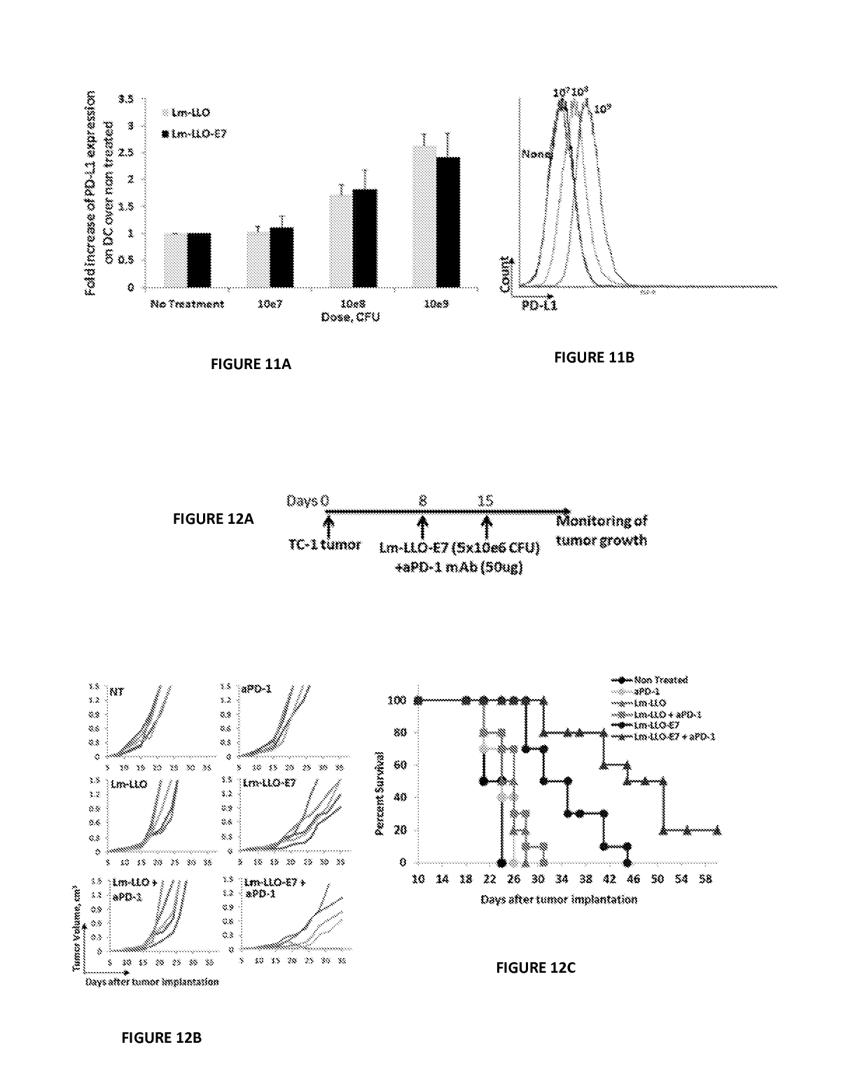

To define the immune mechanism and evaluate the immunologic efficacy of Lm-LLO-E7/anti-PD-1 Ab combination was next assessed to determine the levels of antigen-specific IFNγ-producing cells in spleens from treated tumor bearing mice and tumor-infiltrated CD8 T cells. Mice were implanted with TC-1 cells and treated as described above for therapeutic experiments, except, six days after the second treatment mice were sacrificed and spleens and tumors were harvested. Analysis of E7-specific IFNγ-producing cells was performed using a standard ELISPOT assay. As expected, treatment with Lm-LLO-E7 alone induced significant levels of IFNγ-producing E7-specific cells compared to controls (P<0.001). Notably, addition of PD-1/PD-L1 blockade with anti-PD-1 Ab, to Lm-LLO-E7 resulted in further significant increase in antigen specific immune response when compared to Lm-LLO-E7 alone (P<0.01) (FIG. 13A). To further determine the mechanism by which combining Lm-LLO-E7/anti-PD-1 Ab exerts its therapeutic effect, the influence of treatment on tumor infiltrated CD8 T cells was tested. Tumor-infiltrated CD8 T cells were tested on day 21 post tumor implantation in mice treated as described above. Lm-LLO-E7 and Lm-LLO-E7/anti-PD-1 Ab showed a significant increase in tumor-infiltrated CD8 T cells compared to control groups (P<0.05 for Lm-LLO-E7 alone and P<0.001 for Lm-LLO-E7/anti-PD-1 Ab) (FIG. 13B). Similar to peripheral immune response, addition of anti-PD-1 Ab to Lm-LLO-E7 treatment resulted in significant increase in CD8 T cell tumor infiltration compared to Lm-LLO-E7 alone (P<0.05) (FIG. 13B).

### Example 11: Lm-LLO Treatment Significantly Reduces Both Splenic and Tumor-Infiltrated MDSC and Treg Cells Regardless of Presence of Antigen or Anti-PD-1 Ab

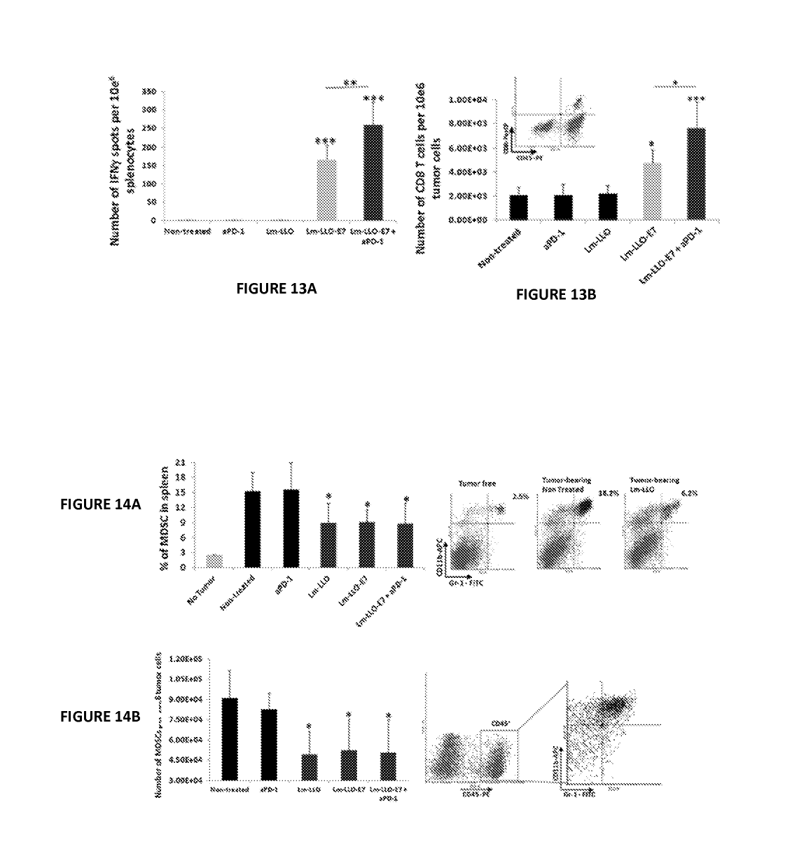

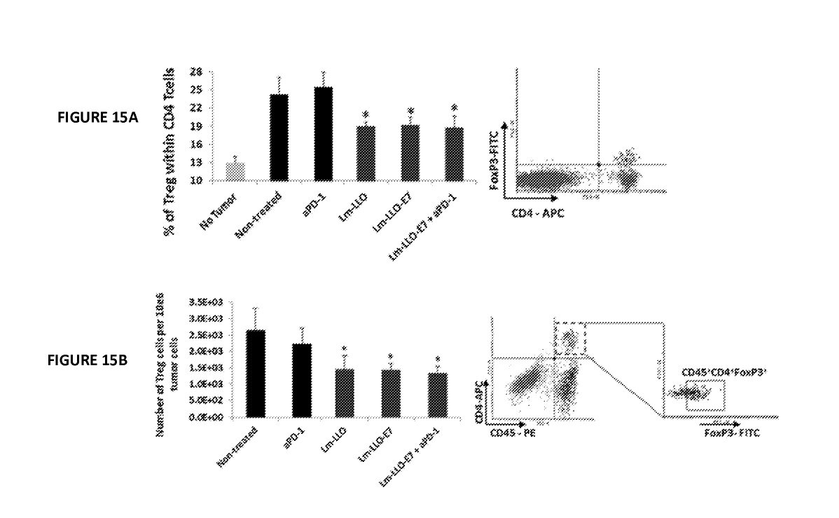

Two cell subsets with profound immune response inhibitory activity are MDSC and Treg cells. Accordingly, these subsets were analyzed both in periphery and within tumor microenvironment to understand the impact of Lm-LLO-E7/anti-PD-1 Ab combinational treatment. Spleens and tumors harvested six days after second vaccination were assessed for percent (spleen) and actual numbers (tumors) of MDSC and Treg cells. While the percent of MDSC in spleens of tumor-free animals is about 2.5%, in presence of tumor this percent significantly increases (˜15%) (FIG. 14A). Surprisingly, treatment with Lm-LLO, regardless of presence of E7 antigen or anti-PD-1 treatment, significantly decreases the levels of MDSC in spleens compared to control animals (P<0.05) (FIG. 14A). Similarly, numbers of tumor-infiltrated MDSC also were significantly decreased after treatment with Lm-LLO, Lm-LLO-E7 and Lm-LLO-E7/anti-PD-1 Ab treatment (FIG. 14B). Importantly, Treg cells in both spleens (FIG. 15A) and tumors (FIG. 15B) were also slightly but significantly decreased in groups treated with Lm-LLO either alone or with E7 or anti-PD-1 Ab.

These data suggest that Lm-LLO is solely responsible for the decrease of MDSC and Tregs in both spleens and tumors of treated mice, and that the addition of antigen or anti-PD-1 antibody does not affect levels of these cells.

### Example 12: Infection of Human DC with Lm-LLO Also Leads to Upregulation of Surface PD-L1 Expression

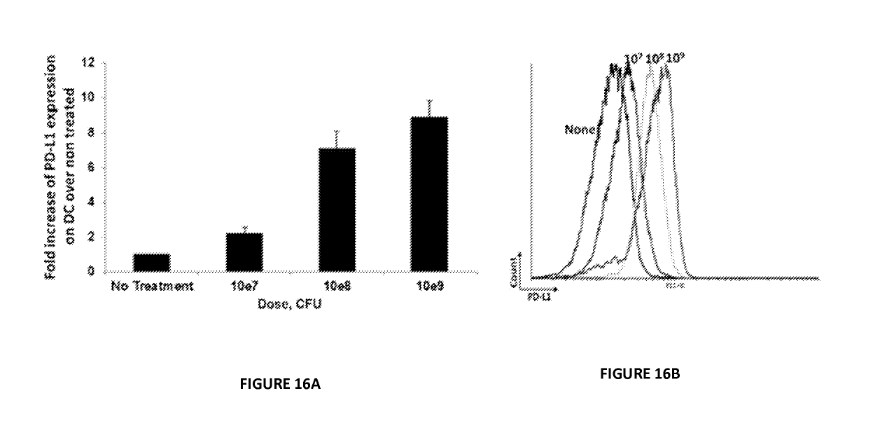

After demonstrating the therapeutic efficacy and immune mechanism by which Lm-LLO-E7/anti-PD-1 Ab combination exerts anti-tumor effect, it was tested if Lm-LLO also affect the levels of PD-L1 expression on human DC and so as, to understand if the findings could be translated into the clinic. Monocyte-derived human DC were isolated from PBMC of healthy volunteers as described in Methods section. Human DC were infected with different concentrations of Lm-LLO and Lm-LLO-E7. It was found that, similar to murine DC, both Lm-LLO and Lm-LLO-E7 infection leads to significant upregulation of surface PD-L1 (FIG. 16A and FIG. 16B and data not shown). As for murine DC, the PD-L upregulation on human DC was dose dependent. This finding suggests that combination of listeria-based vaccine with anti-PD-1 Ab could be a potent and clinically translatable immunotherapeutic approach.

In conclusion, the above findings demonstrate that combination of Lm-LLO-based vaccine with anti-PD-1 Ab leads to increased antigen-specific immune responses and tumor-infiltrating CD8 T cell, decrease in suppressor cells (Treg cells and MDSC) and as a result, leads to significant inhibition of tumor growth and prolonged survival/complete regression of tumors in treated animals. Thus, it was shown that combination of Lm-LLO-based vaccine with blocking of PD-1/PD-L1 interaction is a feasible and translatable approach that can lead to overall enhancement of the efficacy of anti-tumor immunotherapy.

### Example 13: Phase 1/II Study of ADXS11-001 or MEDI4736 Immunotherapies Alone and in Combination, in Patients with Recurrent/Metastatic Cervical or Human Papillomavirus (HPV)-Positive Head and Neck Cancer

Background

Approaches that target key HPV genes critical for cancer growth and metastasis may improve survival for individuals diagnosed with carcinomas of the uterine cervix or head and neck.

ADXS11-001 is a live attenuated Listeria monocytogenes (Lm)-listeriolysin O (LLO) immunotherapy bioengineered to secrete an HPV-E7 tumor antigen as a truncated LLO-E7 fusion protein in cells capable of presenting antigen. This results in HPV-specific T-cell generation, reducing tumor protection in the tumor microenvironment. MEDI4736, an anti-programmed death-1 ligand (PD-L1) antibody, blocks the binding of PD-L1 to PD-1 and CD8, and relieves the inhibition of PD-L-dependent immunosuppressive effects. Inhibition of PD-L1 binding increased the apparent immunologic potency/activity of ADXS11-001 in a preclinical study that showed the combination of ADXS11-11 and an anti-PD-L1 significantly retards tumor growth and prolongs survival in animals.

Methods

This is an open-label, multicenter, 2-part, randomized Phase I/II study (NCT02291055). Patients (≧18 years) with squamous/nonsquamous cervical carcinoma or HPV-associated squamous cell cancer of the head and neck who progressed on ≧1 prior platinum-based therapy in the recurrent/metastatic setting are eligible. The primary objective of Phase I is to evaluate the safety and tolerability of ADXS11-001 plus MEDI4736 and select a recommended Phase II dose (RP2D) for the combination. The primary objective of Phase II is to evaluate the tumor response, progression-free survival (PFS), and safety of ADXS11-001 and MEDI4736 as monotherapy and in combination. Exploratory objectives for both phases will evaluate associations between biomarkers of immunologic response with tumor response and PFS. In Phase I, up to 18 patients receive a fixed dose of ADXS11-001 (1×10⁹ colony-forming units [CFU]), while the dose of MEDI4736 is escalated (starting at 3 mg/kg) according to a standard 3+3 design. In Phase II, patients (nz48) are randomized (1:1:2) to receive either ADXS11-001 (1×10⁹ CFU) or MEDI4736 (10 mg/kg) or both at the RP2D; all treatment arms are stratified by disease. In both phases, ADXS11-001 is administered every 4 weeks and MEDI4736 every 2 weeks. Patients receive treatment up to 1 year or until they discontinue due to disease progression or unacceptable toxicity. Efficacy parameters are evaluated by Response Evaluation Criteria In Solid Tumors (RECIST) and immune-related RECIST criteria, and safety determined using the Common Terminology Criteria for Adverse Events (CTCAE).

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. An immunogenic composition comprising (i) an immune checkpoint inhibitor and/or a T-cell stimulator, and (ii) a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof.

2. (canceled)

3. (canceled)

4. The composition of claim 1, wherein said nucleic acid molecule is integrated into the Listeria genome.

5. The composition of claim 1, wherein said nucleic acid molecule is in a bacterial artificial chromosome in said recombinant Listeria strain.

6. The composition of claim 1, wherein said nucleic acid molecule is in a plasmid in said recombinant Listeria strain.

7. The composition of claim 6, wherein said plasmid is stably maintained in said recombinant Listeria strain in the absence of antibiotic selection.

8. The composition of claim 6, wherein said plasmid does not confer antibiotic resistance upon said recombinant Listeria.

9. The composition of claim 1, wherein said heterologous antigen is a tumor-associated antigen.

10. The composition of claim 9, wherein said tumor-associated antigen is a human papilloma virus (HPV).

11. The composition of claim 9, wherein said tumor-associated antigen is an angiogenic antigen.

12. (canceled)

13. (canceled)

14. The composition of claim 1, wherein said recombinant Listeria comprises a mutation in the endogenous actA virulence gene.

15. The composition of claim 1, wherein said recombinant Listeria comprises a mutation in the endogenous prfA gene.

16. The composition of claim 15, wherein said prfA mutation is a D133V mutation.

17. The composition of claim 14, wherein said recombinant Listeria comprises a mutation in the endogenous D-alanine racemase (dal) and D-amino acid transferase (dat) genes.

18. (canceled)

19. (canceled)

20. The composition of claim 1, wherein said nucleic acid further contains a second open reading frame that encodes a metabolic enzyme.

21. The composition of claim 20, wherein said metabolic enzyme encoded by said second open reading frame is an alanine racemase enzyme or a D-amino acid transferase enzyme.

22. The composition of claim 1, wherein said immune checkpoint inhibitor is a PD-1 signaling pathway inhibitor, a CD-80/86 and CTLA4 signalling pathway inhibitor, a T cell membrane protein 3 (TIM3) signalling pathway inhibitor, an adenosine A2a receptor (A2aR) signalling pathway inhibitor, a lymphocyte activation gene 3 (LAG3) signalling pathway inhibitor, or a killer immunoglobulin receptor (KIR) signalling pathway inhibitor.

23. The composition of claim 22, wherein said PD1 signaling pathway inhibitor is a molecule blocking PD-1 receptor interactions with PD-1 Ligand 1 (PD-L1) and PD-1 Ligand 2 (PD-L2).

24. The composition of claim 23, wherein said molecule blocking PD-1 receptor interactions with PD-1 Ligand 1 (PD-L1) and PD-1 Ligand 2 (PD-L2) is a molecule interacting with PD-1, PD-L1 or PD-L2.

25. The composition of claim 24, wherein said molecule interacting with PD-1 is an anti-PD-1 antibody, a truncated PD-L1 protein, or a truncated PD-L2 protein.

26. (canceled)

27. The composition of claim 25, wherein said truncated PD-L1 protein comprises the cytoplasmic domain of PD-L1 protein.

28. The composition of claim 25, wherein said truncated PD-L2 protein comprises the cytoplasmic domain of PD-L2 protein.

29. The composition of claim 24, wherein said molecule interacting with PD-L1 is an anti-PD-L1 antibody, a truncated PD-1 protein, a PD-1 mimic, or a small molecule that binds PD-L1.

30. The composition of claim 24, wherein said molecule interacting with PD-L2 is an anti-PD-L2 antibody, a truncated PD-1 protein, a PD-1 mimic, or a small molecule that binds PD-L2.

31. The composition of claim 24, wherein said molecule interacting with PD-1 is a truncated PD-1 protein.

32. The composition of claim 31, wherein said truncated PD-1 protein comprises the cytoplasmic domain of PD-1 protein.

33. The composition of claim 1, wherein said T-cell stimulator is an an antigen presenting cell (APC)/T cell agonist.

34. The composition of claim 33, wherein said agonist is a CD134 or a ligand thereof or a fragment thereof, CD-137 or a ligand thereof or a fragment thereof, or an Includible T cell costimulator (ICOS) or a ligand thereof or a fragment thereof.

35. The composition of claim 1, further comprising an adjuvant.

36. The composition of claim 35, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

37. A method of eliciting an enhanced anti-tumor T cell immune response in a subject, said method comprising the step of administering to said subject an immunogenic composition comprising (i) an immune checkpoint inhibitor and/or a T-cell stimulator, and (ii) a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof.

38. (canceled)

39. (canceled)

40. The method of claim 37, wherein said immune response comprises increasing (i) a level of interferon-gamma producing cells or (ii) tumor infiltration by T effector cells.

41. (canceled)

42. The method of claim 40, wherein said T effector cells are CD45+CD8+ T cells or CD4+Fox3P− T cells.

43. The method of claim 37, wherein said immune response comprises a decrease in the frequency of (i) T regulatory cells (Tregs) in the spleen and the tumor microenvironment or (ii) myeloid derived suppressor cells (MDSCs) in the spleen and the tumor microenvironment.

44. (canceled)

45. The method of claim 37 wherein said method further comprises inhibiting tumor-mediated immunosuppression in a subject.

46. (canceled)

47. (canceled)

48. A method for increasing the ratio of T effector cells to regulatory T cells (Tregs) in the spleen and tumor of a subject, said method comprising the step of administering to said subject an immunogenic composition comprising (i) an immune checkpoint inhibitor and/or a T-cell stimulator, and (ii) a recombinant attenuated Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein, or a PEST amino acid sequence fused to a heterologous antigen or fragment thereof.

49. (canceled)

50. (canceled)

51. The method of claim 37, further comprising administering with, prior to, or after said administration of said immunogenic composition a cytokine that enhances said anti-tumor immune response.

52. The method of claim 51, wherein said cytokine is: a type I interferon (IFN-α/IFN-β), TNF-α, IL-1, IL-4, IL-12, INF-γ.

53. The method of claim 37, further comprising administering with, prior to, or after said administration of said immunogenic composition a tumor kinase inhibitor (TKI) that enhances said anti-tumor immune response.

54. The method of claim 53, wherein said TKI is selected from Table 1.

55. The method of claim 37, further comprising administering with, prior to, or after said administration of said immunogenic composition an indoleamine 2,3-dioxygenase (IDO) pathway inhibitor.

56. The method of claim 55, wherein said IDO pathway inhibitor is a small molecule that binds or interacts with IDO, or an anti-IDO antibody.

57. The method of claim 37, wherein the checkpoint inhibitor and/or a T-cell stimulator comprised by said immunogenic composition is administered to the subject before, concurrently with, or after the administration of the recombinant Listeria strain.

58. (canceled)

59. (canceled)

60. A method of claim 37, further comprising the step of administering a booster dose of said immunogenic composition, said recombinant Listeria, said T cell stimulator or said checkpoint inhibitor to said subject.

61. (canceled)

62. (canceled)

63. The method of claim 37, wherein said recombinant Listeria comprises a mutation in the endogenous actA virulence gene and the endogenous D-alanine racemase (dal) and D-amino acid transferase (dat) genes.

64. The method of claim 37, wherein said recombinant Listeria comprises a mutation in the endogenous prfA gene, wherein the prfA mutation is a D133V mutation.

65. The method of claim 37, wherein said heterologous antigen is human papilloma virus (HPV).


SEQUENCE LISTING (14 sequences)

SEQ ID NO: 1 - protein, 32 aa, Artificial Sequence
  PEST amino acid sequence
KENSISSMAPPASPPASPKTPIEKKHADEIDK

SEQ ID NO: 2 - protein, 441 aa, Artificial Sequence
  N-terminal fragment of an LLO protein
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 416 aa, Artificial Sequence
  LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 5 - protein, 14 aa, Listeria monocytogenes
KTEEQPSEVNTGPR

SEQ ID NO: 6 - protein, 28 aa, Listeria monocytogenes
KASVTDTSEGDLDSSMQSADESTPQPLK

SEQ ID NO: 7 - protein, 20 aa, Listeria monocytogenes
KNEEVNASDFPPPPTDEELR

SEQ ID NO: 8 - protein, 33 aa, Listeria monocytogenes
RGGIPTSEEFSSLNSGDFTDDENSETTEEEIDR

SEQ ID NO: 9 - protein, 17 aa, Streptococcus pyogenes
KQNTASTETTTTNEQPK

SEQ ID NO: 10 - protein, 17 aa, Streptococcus equisimilis
KQNTANTETTTTNEQPK

SEQ ID NO: 11 - protein, 9 aa, Artificial Sequence
  E7 peptide
RAHYNIVTF

SEQ ID NO: 12 - protein, 9 aa, Artificial Sequence
  ELISPOT irrelevant peptide
KVPRNQDWL

SEQ ID NO: 13 - DNA, 1620 bp, Artificial Sequence
  tLLO-E7 fusion
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaacca

SEQ ID NO: 14 - protein, 540 aa, Artificial Sequence
  tLLO-E7 fusion
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP